(19) United States
(12) Patent Application Publication
(10) Pub. No.: US 2026/0151352 A1
(43) Pub. Date: Jun. 4, 2026

(54) POLY(AMINE-CO-ESTER) POLYMERIC PARTICLES FOR SELECTIVE PULMONARY DELIVERY

(71) Applicant: YALE UNIVERSITY, New Haven, CT (US)

(72) Inventors: W. Mark Saltzman, New Haven, CT (US); Daniel Greif, Guilford, CT (US); Aglaia Ntokou, New Haven, CT (US); Amy Kauffman, Kennebunkport, ME (US)

(21) Appl. No.: 19/382,438

(22) Filed: Nov. 7, 2025

Related U.S. Application Data
(63) Continuation of application No. 17/332,175, filed on May 27, 2021, now abandoned.
(60) Provisional application No. 63/057,626, filed on Jul. 28, 2020.

(51) Int. Cl. A61K 9/51; A61K 9/00; A61K 35/15; A61K 45/06; B82Y 5/00; B82Y 30/00

(52) U.S. Cl. CPC A61K 9/5153; A61K 9/007; A61K 35/15; A61K 45/06; B82Y 5/00; B82Y 30/00

(57) ABSTRACT

Poly(amine-co-ester) polymers, methods of forming active agent-load polyplexes and particles therefrom, and methods of using them for delivery of nucleic acid agents with optimal uptake have been developed. Examples demonstrate critical molecular weights in combination with exposed carboxylic and/or hydroxyl groups, and methods of making. Typically, the compositions are less toxic, more efficient at drug delivery, or a combination thereof compared to a control other transfection reagents. In some embodiments, the compositions are suitable for in vivo delivery, and can be administered systemically to a subject to treat a disease or condition.

### CROSS-REFERENCE TO RELATED APPLICATIONS

This application is a continuation of copending application Ser. No. 17/332,175, filed May 27, 2021, which claims the benefit of and priority to U.S. Application No. 63/057,626, filed on Jul. 28, 2020, which are hereby incorporated herein by reference in their entireties.

### STATEMENT REGARDING FEDERALLY SPONSORED RESEARCH OR DEVELOPMENT

This invention was made with government support under HL142674, HL133016, and HL150766 awarded by National Institutes of Health. The government has certain rights in the invention.

### FIELD OF THE INVENTION

The field of the invention is generally related to polymer compositions and methods for improved pulmonary delivery of diagnostic, prophylactic and/or therapeutic agents for selective delivery to and uptake of agents by pulmonary immune cells, especially macrophages and monocytes.

### BACKGROUND OF THE INVENTION

Cardiovascular diseases, such as pulmonary hypertension (PH), have a major deleterious impact on human health. Indeed, PH, which is defined by a mean pulmonary arterial pressure greater than 20 mmHg, is responsible for more than 20,000 deaths annually in the United States alone (Simonneau G, et al. Eur Respir J. 2019; 53(1); George Chest. 2014; 146(2):476-95). PH includes a heterogenous collection of clinical conditions that are classified into five groups by the World Health Organization (WHO) based on clinical presentation, hemodynamics, pathological findings and therapies (Simonneau).

WHO Group 1 or pulmonary arterial hypertension (PAH), which includes idiopathic (IPAH; formerly classified as primary PH), and Group 3, which is due to lung diseases and/or hypoxia, are representative. Approximately one-half of PAH cases are IPAH, heritable or drug-induced. Another important subgroup are associated PAH conditions of which the leading cause is connective tissue disease, predominantly systemic sclerosis (SSc; also known as scleroderma) (Hoeper M M, et al. Lancet Respir Med. 2016; 4(4):306-22; Galie N, et al. Eur Heart J. 2016; 37(1):67-119).

Unfortunately, PAH is highly morbid and lethal with 50% of patients dying within seven years of initial diagnosis (Benza Chest. 2012; 142(2):448-56). Furthermore, the prognosis of SSc-PAH is dramatically worse than that of IPAH (Fisher M R, et al. Arthritis Rheum. 2006; 54(9):3043-50). Despite a number of available medications for PAH, no therapies induce reversal or prevent progression of the disease. Similarly, among Group 3 patients, PH portends a substantially worse prognosis for the underlying lung disease (Hoeper 2016).

Many cardiovascular diseases, such as atherosclerosis and arterial restenosis, are characterized by excess and aberrant smooth muscle cells (SMCs), and similarly SMC coating of normally unmuscularized distal pulmonary arterioles in PH is a key pathological feature. This hypermuscularization reduces pulmonary arterial compliance, which is a strong independent predictor of mortality in IPAH (Mahapatra, et al. J Am Coll Cardiol. 2006; 47(4):799-803.). Current treatments for PAH primarily induce vascular dilation, but these therapies do not attenuate the excess muscularization. The treatment gap largely reflects limits in our understanding of pathogenesis, and hence further investigations into the pathobiology of PH are paramount.

Specialized pulmonary arteriole SMCs expressing platelet-derived growth factor receptor (PDGFR)-β clonally expand and give rise to pathological distal arteriole SMCs during hypoxia-induced PH, but regulation of this stereotyped process is incompletely understood (Sheikh Cell Rep. 2014; 6(5):809-17; Sheikh Sci Transl Med. 2015; 7(308):308ra159). Upregulation of hypoxia-inducible factor (HIF) 1-α in SMCs plays a key role in distal muscularization, and in addition to such pathways in SMCs themselves, non-cell autonomous regulation is critical (Ball, et al. Am J Respir Crit Care Med. 2014; 189(3):314-24.; Sheikh Cell Rep. 2018; 23(4):1152-65). In this context, endothelial cells (ECs) are the most highly studied cell type. For instance, the PDGF pathway is integral to vascular SMC development and disease Andrae et al. Genes Dev. 2008; 22(10):1276-312; Seidelmann Cell Mol Life Sci. 2014; 71(11):1977-99), and deletion of the ligand PDGF-β in ECs attenuates hypoxia-induced distal pulmonary arteriole muscularization, PH and right ventricle hypertrophy (RVH) (Sheikh 2018)).

Experimental hypoxia in rodents causes distal pulmonary arteriole muscularization, PH and right ventricle hypertrophy. The signaling pathway regulated by platelet-derived growth factor, abbreviated PDGF, is integral to SMC pathobiology in PH. Indeed, there are increased levels of the receptor PDGFR-β in pathological SMCs, and deletion of the ligand PDGF-β in endothelial cells attenuates PH. Over the last decade, new findings in the involvement of the immune system in several diseases has motivated scientists to investigate further the role of macrophages in lung pathologies. Hypoxia induces increased macrophage recruitment in the lung and pharmacological inhibition of select receptors or agonists expressed by macrophages (e.g., CX3CR1, leukotriene B4) have been shown to mitigate PH; however, these products are also produced by other cell types, raising the issue of cell specificity.

Beyond vascular cell types, immune cells, including monocytes/macrophages, have recently received increasing attention in the context of PH (Florentin et al. Cytokine. 2017; 100:11-5; Nicolls et al Am J Respir Crit Care Med. 2017; 195(10):1292-95). With exposure of mice to hypoxia, monocytes migrate to the lung perivascular space and differentiate into interstitial macrophages (Florentin et al Cytokine. 2017; 100:11-5; Nicolls et al. Am J Respir Crit Care Med. 2017; 195(10):1292-9).

Bronchoalveolar lavage of these mice demonstrates an increase in macrophages in the aspirated bronchoalveolar lavage fluid (BALF) as well as in the residual lung (Amsellem V, et al. Am J Respir Cell Mol Biol. 2017; 56(5):597-608). Similarly, cells expressing the macrophage marker CD68 are enriched in proximity to vascular obstructive lesions in the lungs of human PAH patients (Tuder et al. Am J Pathol. 1994; 144(2):275-85). In rodent models of PH, global genetic or pharmacological inhibition of select receptors or agonists expressed by macrophages (e.g., CX3CR1, leukotriene B4) have been shown to mitigate PH (Amsellem, et al. Sci Transl Med. 2013; 5(200):200ra117); however, these products are produced by other cell types as well, raising the issue of macrophage specificity.

Although monocytes/macrophages are undoubtedly important players in the pathogenesis of PH and other vascular diseases, their roles in regulating the biology of SMCs in these contexts are not well established. It was recently demonstrated that during the formation of atherosclerotic plaques, clonal expansion of rare SMCs is regulated by bone marrow-derived cells (most likely macrophages) (Misra A, et al. Nat Commun. 2018; 9(1):2073). Furthermore, medium conditioned by activated macrophages from atheroprone mice induces aortic SMC migration and proliferation (Misra 2018). Relevant to PH, hypoxia exposure of macrophages pre-activated by interleukin-4 generates conditioned medium that induces proliferation of pulmonary artery SMCs (PASMCs) (Vergadi E, et al. Circulation. 2011; 123(18):1986-95). In addition, dual inhibition of C—C motif chemokine receptor 2 and 5 attenuates macrophage conditioned medium-induction of PASMC proliferation and migration (Abid, et al. Eur Respir J. 2019; 54(4)).

It was also recently found that downregulation of PDGF-B in monocytes/macrophages with the inefficient Csf1r-Cre-Mer-Cre modestly inhibits hypoxia-induced pulmonary vascular remodeling, but hemodynamics and underlying pathways were not assessed (Sheikh, Cell Reports, 2018. 23:1152; Epelman S, et al. Immunity. 2014; 40(1):91-104).

Even if these cells are critical to prevention or treatment of PH, there is no means for selective delivery to these cells, to treat the disease or alleviate the symptoms thereof.

Therefore, it is an object of the invention to provide improved polymers which can selectively and effectively deliver therapeutic, diagnostic, and/or prophylactic agents, agents to pulmonary immune cells, especially pulmonary macrophages and monocytes.

### SUMMARY OF THE INVENTION

Lung macrophage-derived PDGF-B plays a key role in pathological SMC expansion and can be used as a therapeutic target to treat or alleviate diseases such as PH. Studies were conducted using mouse models, cell type-specific deletion of multiple genes, human macrophages from IPAH and SSc-PAH patients and in vivo nanoparticle-delivered siRNA against PDGF-β. Depletion of lung macrophages or PDGI-β deletion in myeloid cells attenuates hypoxia-induced distal muscularization, PH and alveolar myofibroblast accumulation. The results establish that monocytes/macrophages are important players in pulmonary hypertension (PH).

Using a hypoxia mouse model as well as human monocyte-derived macrophages, it was demonstrated that platelet-derived growth factor (PDGF)-β from macrophages is upregulated in PH patients and in the lungs of experimental PH mice. Macrophage-derived PDGF-B induces increased migration and proliferation of human pulmonary artery smooth muscle cells, key components of the pathogenesis of PH. Furthermore, the findings indicate that genetic deletion of PDGF-β in myeloid cells prevents hypoxia-induced PH. The results demonstrate that HIF1-α and HIF2-α are upstream of PDGF-B in macrophages and deletion of Hifa gene in LysM⁺ cells in hypoxia exposed mice has similar effects as PDGF-β deletion. As a complementary approach, under normoxic conditions, HIFα gain-of-function in myeloid cells induces lung macrophage accumulation and PDGF-β expression and distal muscularization, PH and RVH. Medium conditioned by macrophages from IPAH and SSc-PAH patients induce human PASMC (hPASMC) proliferation and migration in a PDGF-B-dependent manner. The results indicate that orotracheally administered nanoparticles loaded with PDGF-β siRNA markedly attenuates hypoxia-induced lung macrophage PDGF-β expression, distal muscularization, PH, RVH and alveolar myofibroblast accumulation. These all demonstrate targeting lung macrophage-derived PDGF-B as a therapeutic strategy for PH.

A number of nanoparticle-based technologies are currently FDA-approved, but they are predominantly administered intravenously to reach the target organ(s) via the circulation. It has been discovered that particles formed of poly(amine-co-ester) polymers can be used for selective delivery of therapeutic, prophylactic or diagnostic agents to, for uptake by, immune cells lining the pulmonary tract, such as macrophages. Examples demonstrate that the particles have high loading and selective uptake in the absence of targeting moieties, when administered to the pulmonary tract.

In addition to pulmonary disorders such as PH, diseases or condition to be treated include infectious diseases, cancers, metabolic disorders, autoimmune diseases, inflammatory disorders, and age-related disorders. The particles can be administered by aerosol, inhaler, dry powder, intubation and instillation.

Examples demonstrate orotracheally administered large nanoparticles (400 nm in diameter) loaded with silencing (si) RNA against PDGF-β to mice. These nanoparticles are preferentially taken up by lung macrophages (of the total cells that take up nanoparticles, the percentage of cells that are macrophages are ^{˜}95% in the bronchoalveolar lavage fluid and ^{˜}85% in the residual lung following bronchoalveolar lavage). With orotracheal administration, the efficiency of PDGF-β silencing is high in lung macrophages (>85% knockdown) and can effectively prevent/abrogate hypoxia-induced pathological distal arteriole muscularization, pulmonary artery pressure and right ventricle hypertrophy.

### BRIEF DESCRIPTION OF THE DRAWINGS

FIGS. 1A-1F are graphs showing lung macrophages accumulate with hypoxia and are critical for hypoxia-induced pulmonary vascular remodeling and PH. Wild type mice were exposed to hypoxia (10% FiO₂) for up to 21 days or maintained in normoxia as indicated. BALF and residual lung were harvested, and single cell suspensions were subjected to flow cytometric analysis. The percentage of total cells in the given compartment that are CD64⁺Ly6G⁻ macrophages was determined. n=3 mice per time point. FIGS. 1A and 1B are graphs of CD64+Ly6G− cells (%) over days of hypoxia, for BALF (FIG. 1A) and residual lung (FIG. 1B). FIGS. 1C-1D are graphs of RVSP (mm Hg) (FIG. 1C) and RV/(LV+S) (FIG. 1D, Fulton index (F; weight ratio of the right ventricle [RV] to sum of the left ventricle [LV] and septum [S]) are shown. n=3 mice.) for normoxia and hypoxia. Liposomes containing PBS (vehicle) or clodronate were administered orotracheally at the onset of hypoxia (or normoxia as a control) and every 3 days thereafter during the 21-day treatment. FIGS. 1E and 1F, the percent of CD64⁺Ly6G⁻ macrophages in total cells of the BALF (FIG. 1G) and residual lung (FIG. 1H) was determined. n=3 mice.

FIGS. 2A-2F are graphs showing lung macrophage PDGF-β levels increase with hypoxia, and PDGF-β deletion in LysM⁺ cells attenuates distal muscularization and PH. BALF (FIG. 2A) and residual lung (FIG. 2B) CD64⁺Ly6G⁻ cells were isolated by FACS from wild type mice exposed to hypoxia (10% FiO₂) for up to 21 days or normoxia as indicated. PDGF-mRNA levels were measured by qRT-PCR (see Table 1). n=3 mice per time point with qRT-PCR done in triplicate. FIGS. 2C-2F, PDGF-β^{(flox/flox)} mice also carrying no Cre or LysM-Cre were exposed to hypoxia for 21 days or maintained in normoxia. FIG. 2C, RVSP; FIG. 2D, RV/(LV+S), FIG. 2E, change in RV/LV+S, and FIG. 2F, Myofibs/100 alveoli. The Fulton index differences between hypoxia and normoxia values stratified by genotype are displayed in FIG. 2C. One-way ANOVA with Tukey's multiple comparison test (*, **, ***, #, vs. normoxia, p<0.05, <0.01, <0.001, <0.0001, respectively) was used in (FIGS. 2C-2D), and Student's t-test was used in FIG. 2E.

FIGS. 3A-3D. Vhl deletion in LysM⁺ cells induces distal muscularization and PH under normoxia. Vhl^{(flox/flox)} mice also carrying no Cre or LysM-Cre were maintained in normoxia for 49 days after birth. FIG. 3A, BALF was isolated and PDGF-β transcript levels were measured by Fulton index (FIG. 3C, BALF; 3B, lung) are shown. The number of macrophages (asterisks) quantified per 100 alveoli in (FIG. 3D). More than 500 alveoli per mouse were quantified. n=3 mice. Student's t-test was used.

FIGS. 4A-4F. Hif1a deletion in myeloid cells attenuates hypoxia-induced PDGF-β expression, distal muscularization and PH. BALF cells were isolated from normoxic or hypoxic (10% FiO₂, up to 21 days) wild type mice. HIF1-α and β-actin protein were assessed by Western blot with densitometry of HIF1-αrelative to β-actin. n=3 mice per time point. One-way ANOVA with Tukey's multiple comparison test. Hif1α^{(flox/flox)} mice also carrying no Cre or LysM-Cre were exposed to hypoxia for 3 or 21 days. At hypoxia day 3, PDGF-β transcript levels of BALF cells were determined by qRT-PCR (FIG. 4A, 4B). Lung vibratome sections were stained for SMA, macrophage marker CD64 and nuclei (DAPI). The number of macrophages and alveolar myofibroblasts were quantified per 100 alveoli (FIGS. 4C, 4D). n=3-5 mice, qRT-PCR was done in triplicate. More than 700 alveoli were quantified per mouse. At hypoxia day 21, vibratome sections with distal arterioles in the L.L1.A1.L1 area were stained for SMA and CD31, and RVSP and the Fulton index were measured as shown in FIG. 4E, 4F. n=3 mice.

FIGS. 5A-5F. Deletion of Hif2a in LysM⁺ cells attenuates hypoxia-induced PDGF-β expression, distal muscularization and PH. BALF cells were isolated from wild type mice exposed to normoxia or hypoxia (10% FiO₂) for up to 21 days. Western blot was used to assess HIF2-α and β-actin protein levels with densitometry of HIF2-α relative to β-actin. n=3 mice per time point. FIG. 5A. One-way ANOVA with Tukey's multiple comparison test. FIGS. 5B-5F. Hif2α^{(flox/flox)} mice also carrying no Cre or LysM-Cre were exposed to hypoxia for 3 or 21 days. At hypoxia day 3, BALF cells were isolated with PDGF-β mRNA levels determined by qRT-PCR (FIG. 5B), and vibratome sections of the lung were stained for SMA, CD64 and nuclei (DAPI) The number of macrophages and alveolar myofibroblasts were quantified per 100 alveoli (FIGS. 5C-5D). n=3-5 mice, qRT-PCR was done in triplicate. More than 700 alveoli were quantified per mouse. At hypoxia day 21, vibratome sections with distal arterioles in the L.L1.A1.L1 area were stained for SMA and MECA-32 and RVSP and the Fulton index were measured (FIGS. 5E, 5F). n=3 mice. Student's t-test was.

FIGS. 6A-6E. PDGF-B secreted by macrophages from PAH patients promotes hPASMC proliferation and migration. Monocytes were isolated from peripheral blood mononuclear cells of human controls and IPAH or SSc-PAH patients and differentiated into macrophages in culture. FIG. 6A, Macrophages derived from human control monocytes were cultured under normoxic or hypoxic (3% O₂) conditions for 12 h, and then PDGF-β mRNA levels were measured by qRT-PCR. n=3 humans (two females and one male, aged 30-60 years old) with qRT-PCR done in triplicate. FIG. 6B, qRT-PCR was used to assay PDGF-β mRNA levels of macrophages from controls and PAH patients. n=5 humans per PAH diagnostic class and n=9 controls (see Table S2) with qRT-PCR done in triplicate. FIG. 6C, hPASMCs were cultured for 24 h with medium preconditioned by control and patient macrophages. BrdU was included in the last 10 h of this incubation. Cells were then stained for BrdU and nuclei (propidium iodide [PI]). In FIG. 6C, the percent of total cells (PI nuclei) expressing BrdU for control humans and patients was normalized to this percentage for controls. In FIG. 6D, anti-PDGF-B blocking antibody or control IgG was added to the conditioned medium 1 h prior to incubation with hPASMCs. Results are the ratio of the percent of total (PI⁺) cells that are BrdU⁺ for anti-PDGF-B treatment relative to IgG treatment, stratified by patient diagnostic class. n=3 humans per PAH diagnostic class and n=6 controls (see Table S3), 10 microscopic fields per human, 30-60 cells per field. Medium preconditioned by control or patient macrophages was treated with anti-PDGF-B blocking or control IgG antibody for 1 h and then placed in the bottom chamber of a Boyden apparatus. hPASMCs were added to the top chamber to assess migration toward the conditioned medium for 8 h. Migrated cells (i.e., on the membrane's bottom surface) were stained with Crystal Violet. In FIG. 6E, quantification of the migrated cells relative to control patients, IgG treatment is shown. n=4 humans per PAH class and n=3 controls (see Table 4), 5 microscopic fields per human, 8-90 cells per field. One-way ANOVA with Tukey's multiple comparison test and Student's t-test were used. #, ##vs. IPAH, p<0.05, <0.01, and *, **, ***, ns vs. corresponding IgG controls, p<0.05, <0.01, <0.0001 not significant, respectively.

FIGS. 7A-7F. Nanoparticle-mediated knockdown of PDGF-β attenuates distal arteriole muscularization, myofibroblast accumulation and PH. Nanoparticles (diameter 400 nm) loaded with the dye DiD were administered orotracheally to normoxic mice, and 12 h later, cells from BALF and residual lung were stained for CD64 and subjected to flow cytometric analysis. FIG. 7A, Quantification showing the percentage of BALF or residual lung (RL) cells containing DiD⁺ nanoparticles (diameter 400 or 200 nm as indicated) that express CD64. n=3 mice per treatment. BALF cells were harvested from normoxic mice, cultured with DiD-loaded 400 nm nanoparticles for 6 h and then stained for nuclei (DAPI). FIGS. 7B-7F, Nanoparticles of 400 nm diameter were loaded with siRNA targeted against PDGF-β or scrambled (Scr) RNA and then administered to mice at the onset of hypoxia and twice per week thereafter. Lungs were isolated from mice at hypoxia day 3, stained for Ly6G and CD64 and subjected to flow cytometry, and the percent of CD64⁺Ly6G⁻ macrophages was quantified in FIG. 7B. n=3 mice per treatment. In FIG. 7C, PDGF-β RNA levels of CD64⁺Ly6G⁻ macrophages were quantified by qRT-PCR. n=3 mice per treatment with qRT-PCR done in triplicate. In FIGS. 7D-7F, mice were treated with hypoxia for 21 days or maintained in normoxia. For hypoxic mice, sections containing distal arterioles in the L.L1.A1 area or alveolar region were stained for CD31 and SMA. RVSP (FIG. 7D), Fulton index (FIG. 7E) and number of myofibroblasts per 100 alveoli were measured (FIG. 7F). More than 500 alveoli per mouse were quantified. One-way ANOVA with Tukey's multiple comparison test and Student's t-test were used. * vs. normoxia, p<0.05. ns, not significant. Scale bars, 10 μm (D) and 25 μm (I, L).

FIG. 8 is a schematic of the methods used for the animal and human studies.

### DETAILED DESCRIPTION OF THE INVENTION

### I. Definitions

The term “polyplex” as used herein refers to polymeric micro- and/or nanoparticles or micelles typically having encapsulated therein, dispersed within, and/or associated with the surface of, one or more polynucleotides.

The term “microparticles” includes objects having an average diameter from about one or greater microns up to about 1000 microns. The term “microparticles” includes microspheres and microcapsules, as well as structures that may not be readily placed into either of the above two categories. A microparticle may be spherical or nonspherical and may have any regular or irregular shape. Structures with an average diameter of less than about one micron (1000 nm) in diameter, are referred to as “nanoparticles” and include “nanosphere,” and “nanocapsules,” The term “diameter” is used to refer to either the physical diameter or the hydrodynamic diameter. The diameter of an essentially spherical particle may refer to the physical or hydrodynamic diameter. The diameter of a nonspherical particle may refer to the hydrodynamic diameter. As used herein, the diameter of a non-spherical particle may refer to the largest linear distance between two points on the surface of the particle. When referring to multiple particles, the diameter of the particles typically refers to the average diameter of the particles. Particle diameter can be measured using a variety of techniques in the art including, but not limited to, dynamic light scattering and confocal microscopy.

A composition containing microparticles or nanoparticles may include particles of a range of particle sizes. In certain embodiments, the particle size distribution may be uniform, e.g., within less than about a 20% standard deviation of the mean volume diameter, and in other embodiments, still more uniform, e.g., within about 10%, 8%, 5%, 3%, or 2% of the median volume diameter.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

The term “biodegradable” as used herein means that the materials degrades or breaks down into its component subunits, typically by hydrolysis or enzymatic action.

The term “surfactant” as used herein refers to an agent that lowers the surface tension of a liquid.

“Sustained release” as used herein refers to release of a substance over an extended period of time in contrast to a bolus type administration in which the entire amount of the substance is made biologically available at one time.

The phrases “parenteral administration” and “administered parenterally” are art-recognized terms, and include modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intraarticular, subcapsular, subarachnoid, intraspinal and intrastemal injection and infusion.

The term “targeting moiety” as used herein refers to a moiety that localizes to or away from a specific locale. The moiety may be, for example, a protein, nucleic acid, nucleic acid analog, carbohydrate, or small molecule. Said entity may be, for example, a therapeutic compound such as a small molecule, or a diagnostic entity such as a detectable label.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. All integer values of the number of backbone carbon atoms between one and 30 are contemplated and disclosed for the straight chain or branched chain alkyls. Likewise, preferred cycloalkyls have from 3-10 carbon atoms in their ring structure, and more preferably have 5, 6, or 7 carbons in the ring structure. All integer values of the number of ring carbon atoms between three and 10 are contemplated and disclosed for the cycloalkyls.

The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

“Aryl”, as used herein, refers to C₅-C₁₀-membered aromatic, heterocyclic, fused aromatic, fused heterocyclic, biaromatic, or bihetereocyclic ring systems. In some forms, the ring systems have 3-50 carbon atoms. Broadly defined, “aryl”, as used herein, includes 5-, 6-, 7-, 8-, 9-, 10- and 24-membered single-ring aromatic groups that may include from zero to four heteroatoms, for example, benzene, naphthalene, anthracene, phenanthrene, chrysene, pyrene, corannulene, coronene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

“Alkoxy” refers to an alkyl group as defined above with the indicated number of carbon atoms attached through an oxygen bridge. Examples of alkoxy include, but not limited to, methoxy, ethoxy, n-propoxy, i-propoxy, n-butoxy, s-butoxy, n-pentoxy, s-pentoxy, and derivatives thereof.

Primary amines arise when one of three hydrogen atoms in ammonia is replaced by a substituted or unsubstituted alkyl or a substituted or unsubstituted aryl group. Secondary amines have two organic substituents (substituted or unsubstituted alkyl, substituted or unsubstituted aryl or combinations thereof) bound to the nitrogen together with one hydrogen. In tertiary amines, nitrogen has three organic substituents.

“Substituted”, as used herein, means one or more atoms or groups of atoms on the monomer has been replaced with one or more atoms or groups of atoms which are different than the atom or group of atoms being replaced. In some embodiments, the one or more hydrogens on the monomer is replaced with one or more atoms or groups of atoms. Examples of functional groups which can replace hydrogen are listed above in the definition. In some embodiments, one or more functional groups can be added which vary the chemical and/or physical property of the resulting monomer/polymer, such as charge or hydrophilicity/hydrophobicity, etc. Exemplary substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

Unless otherwise indicated, the disclosure encompasses conventional techniques of molecular biology, microbiology, cell biology and recombinant DNA, which are within the skill of the art. See, e.g., Sambrook and Russell, Molecular Cloning: A Laboratory Manual, 3rd edition (2001); Current Protocols In Molecular Biology [(Ausubel, et al. eds., (1987)]; Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)].

### II. Particles

Particles for efficient and selective delivery to the lungs are typically formed of biodegradable biocompatible polymers. These are typically nanoparticles less than 1000 nm, more preferably less than 500 nm, most preferably at least 100 nm. Examples demonstrates that nanoparticles between 200 and 400 nm selectively target pulmonary immune cells such as monocytes and macrophages.

### Polymers

Polymers including poly(amine-co-ester), poly(amine-co-amide), or a combination thereof, and polyplexes and solid core particles formed therefrom. Poly(amine-co-ester) are discussed in WO 2013/082529, WO 2017/151623, WO 2017/197128, U.S. Published Application No. 2016/0251477, U.S. Published Application No. 2015/0073041, and U.S. U.S. Pat. No. 9,272,043.

When substituting the diester monomer in the polymers with diacid, such as sebacic acid, polymers with a mixture of hydroxyl and carboxyl end groups can be obtained. Both of these two end groups can be activated with 1,1′-carbodiimidazole. The activated product can react with amine-containing molecules to yield polymers with new end groups.

The polymers can be further hydrolyzed to release more active end groups, such as —OH and —COOH, both of which can originate from hydrolysis of ester bonds in the polymers (also referred to herein as “actuation”), typically by incubating the polymers, e.g., at a control temperature (e.g., 37° C. or 100° C.), for days or weeks. In some embodiments, the polymers are not hydrolyzed, and thus can be referred to as “non-actuated.”

In some embodiments, the content of a hydrophobic monomer in the polymer is increased relative the content of the same hydrophobic monomer when used to form polyplexes. Increasing the content of a hydrophobic monomer in the polymer forms a polymer that can form solid core nanoparticles in the presence of nucleic acids, including RNAs.

Unlike polyplexes, these particles are stable for long periods of time during incubation in buffered water, or serum, or upon administration (e.g., injection) into animals. They also provide for a sustained release of nucleic acids (e.g., siRNA) which leads to long term activity (e.g., siRNA mediate-knockdown).

### A. Polymer Structure

Poly(amine-co-ester)s or poly(amine-co-amide)s are described herein. In some forms, the polymer has a structure as shown in Formula I:

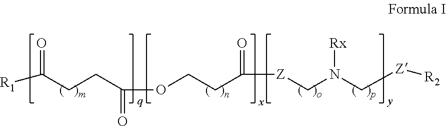

wherein n is an integer from 1-30,
m, o, and p are independently integers from 1-20,
x, y, and q are independently integers from 1-1000,
Rₓ is hydrogen, substituted or unsubstituted alkyl, or substituted or unsubstituted aryl, or substituted or unsubstituted alkoxy,
Z and Z′ are independently O or NR′, wherein R′ is hydrogen, substituted or unsubstituted alkyl, or substituted or unsubstituted aryl,
R₁ and R₂ are chemical entities containing a hydroxyl group, a primary amine group, a secondary amine group, a tertiary amine group, or combinations thereof.

Examples of Rₓ and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

In particular embodiments, the values of x, y, and/or q are such that the weight average molecular weight of the polymer is greater than 20,000 Daltons, greater than 15,000 Daltons, greater than 10,000 Daltons, greater than 5,000 Daltons, greater than 2,000 Daltons. In some forms, the weight average molecular weight of the polymer is between about 2,000 Daltons and about 20,000 Daltons, more preferably between about 5,000 Daltons and about 10,000 Daltons.

The polymer can be prepared from one or more lactones, one or more amine-diols (Z and Z′=O), triamines (Z and Z′=NR′), or hydroxy-diamines (Z═O and Z′=NR′, or Z═NR′ and Z′=O) and one or more diacids or diesters. In those embodiments where two or more different lactone, diacid or diester, and/or triamine, amine-diol, or hydroxy-diamine monomers are used, the values of n, o, p, and/or m can be the same or different.

In some forms, the percent composition of the lactone unit is between about 10% and about 100%, calculated lactone unit vs. (lactone unit+diester/diacid). Expressed in terms of a molar ratio, the lactone unit vs. (lactone unit+diester/diacid) content is between about 0.1 and about 1, i.e., x/(x+q) is between about 0.1 and about 1. Preferably, the number of carbon atoms in the lactone unit is between about 10 and about 24, more preferably the number of carbon atoms in the lactone unit is between about 12 and about 16. Most preferably, the number of carbon atoms in the lactone unit is 12 (dodecalactone), 15 (pentadecalactone), or 16 (hexadecalactone).

In some forms, Z is the same as Z′.

In some forms, Z is O and Z′ is O. In some forms, Z is NR′ and Z′ is NR′. In some forms, Z is O and Z′ is NR′. In some forms, Z is NR′ and Z′ is O.

In some forms, Z′ is O and n is an integer from 1-24, such as 4, 10, 13, or 14. In some forms, Z is also O.

In some forms, Z′ is O, n is an integer from 1-24, such as 4, 10, 13, or 14, and m is an integer from 1-10, such as 4, 5, 6, 7, or 8. In some forms, Z is also O.

In some forms, Z′ is O, n is an integer from 1-24, such as 4, 10, 13, or 14, m is an integer from 1-10, such as 4, 5, 6, 7, or 8, and o and p are the same integer from 1-6, such 2, 3, or 4. In some forms, Z is also O.

In some embodiments, Z′ is O, n is an integer from 1-24, such as 4, 10, 13, or 14, m is an integer from 1-10, such as 4, 5, 6, 7, or 8, and R is alkyl, such a methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, and n-octyl, or aryl, such as phenyl, naphthalyl, anthracenyl, phenanthryl, chrysenyl, pyrenyl, tolyl, or xylyl. In some forms, Z is also O.

In some forms, n is 14 (e.g., pentadecalactone, PDL), m is 7 (e.g., sebacic acid), o and p are 2 (e.g., N-methyldiethanolamine, MDEA).

In some embodiments, the polyplexes or particles are formed from polymer wherein R1 and/or R2 are not relative to corresponding polyplexes wherein R1 and/or R2 consist of or include

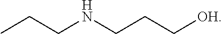

In some embodiments, polyplexes or particles formed from the polymer show improved loading, improved cellular transfection, improved intracellular endosomal release, or a combination thereof of a nucleic acid cargo, such as RNA, more particularly mRNA, relative to corresponding polyplexes wherein R1 and/or R2 consist of or include

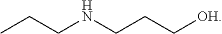

In some forms, the polymer has a structure of Formula II.

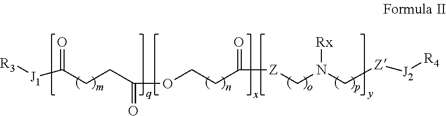

wherein J₁ and J₂ are independently linking moieties or absent,
R₃ and R₄ are independently substituted alkyl containing a hydroxyl group, a primary amine group, a secondary amine group, a tertiary amine group, or combinations thereof. In some forms, the molecular weight of R₃, R₄ or both are at or below 500 Daltons, at or below 200 Daltons, or at or below 100 Daltons.

In some forms, J₁ is —O— or —NH—.

In some forms, J₂ is —C(O)NH— or —C(O)O—.

In some forms, R₃ is identical to R₄.

Preferably, R₃ and/or R₄ are linear.

In some forms, R₃, R₄ or both contain a primary amine group. In some forms, R₃, R₄ or both contain a primary amine group and one or more secondary or tertiary amine groups.

In some forms, R₃, R₄ or both contain a hydroxyl group. In some forms, R₃, R₄ or both contain a hydroxyl group and one or more amine groups, preferably secondary or tertiary amine groups. In some forms, R₃, R₄ or both contain a hydroxyl group and no amine group.

In some forms, at least one of R₃ and R₄ does not contain a hydroxyl group.

In some forms, R₃, R₄ or both are -unsubstituted C₁-C₁₀ alkylene-Aq-unsubstituted C₁-C₁₀ alkylene-Bq, -unsubstituted C₁-C₁₀ alkylene-Aq-substituted C₁-C₁₀ alkylene-Bq, -substituted C₁-C₁₀ alkylene-Aq-unsubstituted C₁-C₁₀ alkylene-Bq, or -substituted C₁-C₁₀ alkylene-Aq-substituted C₁-C₁₀ alkylene-Bq, wherein Aq is absent or —NR₅—, and Bq is hydroxyl, primary amine, secondary amine, or tertiary amine, wherein R₅ is hydrogen, substituted or unsubstituted alkyl, or substituted or unsubstituted aryl.

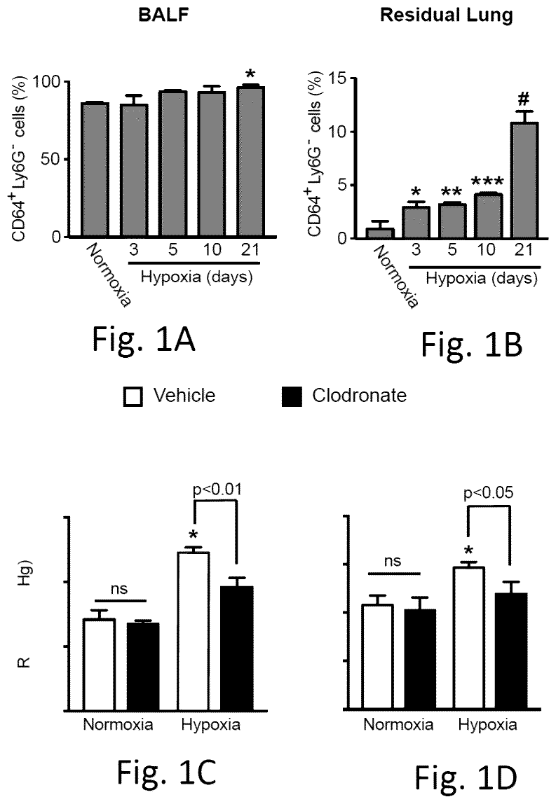

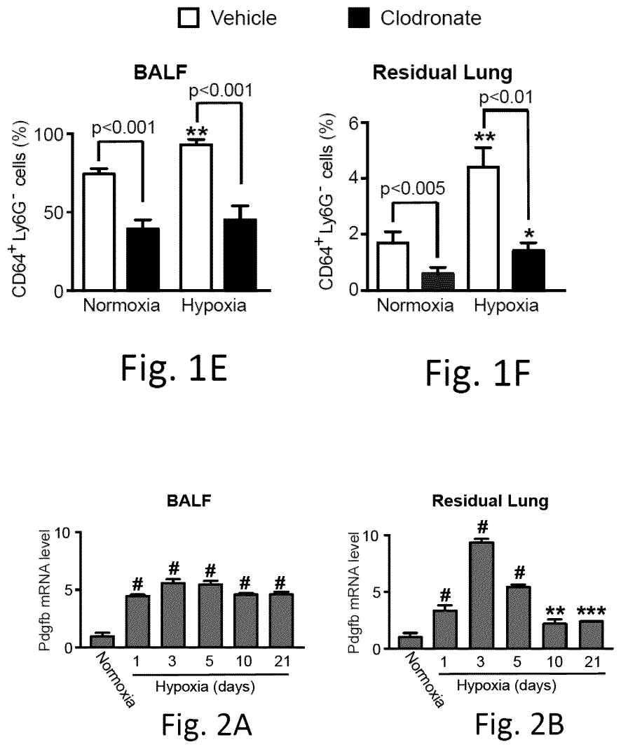

In some forms, R₃, R₄ or both are selected from the groups shown in FIG. 1.

In some forms, the polymer has a structure of Formula III.

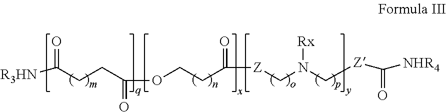

The monomer units can be substituted at one or more positions with one or more substituents. Exemplary substituents include, but are not limited to, alkyl groups, cyclic alkyl groups, alkene groups, cyclic alkene groups, alkynes, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The polymer is preferably biocompatible. Readily available lactones of various ring sizes are known to possess low toxicity: for example, polyesters prepared from small lactones, such as poly(caprolactone) and poly(p-dioxanone) are commercially available biomaterials which have been used in clinical applications. Large (e.g., C₁₆-C₂₄) lactones and their polyester derivatives are natural products that have been identified in living organisms, such as bees. Lactones containing ring carbon atoms between 16 and 24 are specifically contemplated and disclosed.

In some forms, the polymers can be further activated via temperature-controlled hydrolysis, thereby exposing one or more activated end group(s). The one or more activated end group(s) can be, for example, hydroxyl or carboxylic acid end groups, both of which can be generated via hydrolysis of ester bonds within the polymers. The activated polymers can have a weight-average molecular weight between about 5 and 25 kDa, preferably between about 5 and 10 kDa. As used herein, the term “about” is meant to minor variations within acceptable parameters. For the sake of clarity, “about” refers to +10% of a given value. In some forms, the activated polymers contains R₁ or R₂ at one end, and a hydroxyl or carboxylic acid end group at the other end, generated via hydrolysis.

In some forms, the polymer has a structure of Formula IV.

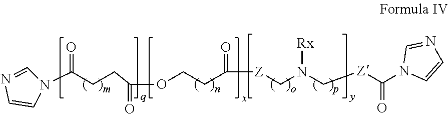

In some forms, the polymer has a structure of Formula V.

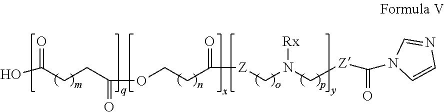

In some forms, the polymer has a structure of Formula VI.

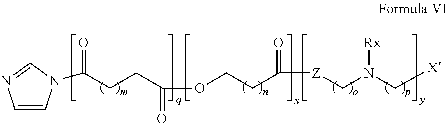

wherein X′ is —OH or —NHR′.

Formulas VI, V, and VI are structures of intermediary products. They can be used to synthesize a wide variety of polymers with a structure of Formula I, II or III.

### B. PEG-Blocking Containing Polymers

The polymers can be used for drug delivery, for example, in the formation of particles, such as microparticles or nanoparticles, or micelles which can release one or more therapeutic, prophylactic, and/or diagnostic agents in a controlled release manner over a desirable period of time.

pH-responsive micelle nanocarriers are often formed via self-assembly of amphiphilic block copolymers and consist of a hydrophilic (e.g. PEG) outer shell and a hydrophobic inner core capable of response to medium pH. Typically, upon changing the medium pH from neutral or slightly basic to mildly acidic, the micelle cores undergo accelerated degradation, become completely soluble in water, or swell substantially in aqueous medium. As the result, the drug-encapsulated micelles with a slow drug-release rate at the physiological pH can be triggered by an acidic pH to rapidly unload the drug molecules. The polymer segments constituting the micelle cores in previous reports include poly(ortho esters), poly(β-amino esters), poly(L-histidine), and others. The major disadvantages with most of the previous micelle systems are the multiple steps required for preparing the copolymers and the difficulty of controlling the polymer molecular weight and adjusting the polymer composition during the copolymer synthesis.

The copolymers exhibited variation in the rate of release as a function of pH. In vitro drug release behaviors of the DTX-encapsulated micelles of PEG2K-PPMS copolymer samples (PEG2K-PPMS-11% PDL, PEG2K-PPMS-30% PDL, and PEG2K-PPMS-51% PDL) were studied in PBS solution at both physiological pH of 7.4 and acidic pH of 5.0. In general, the DTX release from all micelle samples followed biphasic release kinetics and exhibited remarkable pH-dependence. The DTX-loaded PEG2K-PPMS copolymer micelles release 25-45% drug rapidly during the initial 12 h, followed by a more gradual release of additional 25-40% drug for the subsequent 132 h. The influence of the medium pH on the drug release rate is substantial. For example, at the end of the incubation period (144 h), the values of accumulated DTX released from the micelles of PEG2K-PPMS-11% PDL, PEG2K-PPMS-30% PDL, and PEG2K-PPMS-51% PDL copolymers are respectively 66%, 60%, and 55% at physiological pH of 7.4, which increase correspondingly to 85%, 81%, and 75% at acidic pH of 5.0. The observed pH-triggered acceleration of DTX release from the PEG2K-PPMS copolymer micelles is consistent with the earlier observation that changing of the medium pH from 7.4 to 5.0 causes significant swelling of the micelles due to the protonation and size increase of the micelle PPMS cores. This pH-triggered micelle size expansion would certainly facilitate the diffusion and release of entrapped DTX from the micelle cores to the aqueous medium. At a given pH, the DTX release rate is presumably controlled by the interactions between the drug and the PPMS matrix in the micelle cores. Since PDL-rich PEG2K-PPMS copolymers are expected to form strong hydrophobic domains in their micelle inner cores to better trap and retain hydrophobic DTX molecules, the drug release from such copolymer micelles should be more gradual and sustained. This hypothesis is supported by the experimental result showing that at both pH of 7.4 and 5.0, the DTX release rate from PEG2K-PPMS copolymer micelles decreases with increasing PDL content in the PPMS chain segments of the copolymer.

It is known that upon uptake of micelles by tumor cells, the micelle particles are subjected to entrapment in endosomes with pH ranging from 5.5 to 6.0 and in lysosomes with pH ranging from 4.5 to 5.0. As the above results clearly show, these acidic environments would inevitably trigger fast DTX release from PEG2K-PPMS copolymer micelles, thus enhancing the cytotoxicity of the drug-loaded micelles. The amino groups in the copolymers would act as proton sponges to facilitate endosomal escape. Therefore, the pH-responsive properties exhibited by the PEG2K-PPMS copolymer micelles are highly desirable, which render them to be superior carriers for delivery of anticancer drugs.

### C. Methods of Making the Polymers

The polymers are generally modified from synthetic polymers. Exemplary synthetic polymers include poly(amine-co-ester), formed of a lactone, a dialkyl acid, and a dialkyl amine. Methods for the synthesis of poly(amine-co-ester) from a lactone, a dialkyl acid, and a dialkyl amine using an enzyme catalyst, such as a lipase, are also provided. Exemplary lactones are disclosed in U.S. Patent Publication No. US20170121454.

D. Particles Formed from the Polymers

The polymers can be used to prepare micro- and/or nanoparticles having encapsulated therein one or more therapeutic, diagnostic, or prophylactic agents. The agent can be encapsulated within the particle, dispersed within the polymer matrix that forms the particle, covalently or non-covalently associated with the surface of the particle or combinations thereof.

The rate of release can be controlled by varying the monomer composition of the polymer and/or the molecular weight of the polymer and thus the rate of degradation. For example, if simple hydrolysis is the primary mechanism of degradation, increasing the hydrophobicity of the polymer may slow the rate of degradation and therefore increase the time period of release. In all case, the polymer composition is selected such that an effective amount of nucleic acid(s) is released to achieve the desired purpose/outcome.

### E. Perplexes and Micelles

It has been discovered that the gene delivery ability of polycationic polymers is due to multiple factors, including polymer molecular weight, hydrophobicity, and charge density. Many synthetic polycationic materials have been tested as vectors for non-viral gene delivery, but almost all are ineffective due to their low efficiency or high toxicity. Most polycationic vectors described previously exhibit high charge density, which has been considered a major requirement for effective DNA condensation. As a result, they are able to deliver genes with high efficiency in vitro but are limited for in vivo applications because of toxicity related to the excessive charge density.

High molecular weight polymers, particularly terpolymers, have a low charge density. In addition, their hydrophobicity can be varied by selecting a lactone comonomer with specific ring size and by adjusting lactone content in the polymers. High molecular weight and increased hydrophobicity of the lactone-diester-amino diol terpolymers compensate for the low charge density to provide efficient gene delivery with minimal toxicity.

In preferred embodiments, the terpolymers exhibit efficient gene delivery with reduced toxicity. The terpolymers can be significantly more efficient the commercially available non-viral vectors. For examples, the terpolymers can be more than 100× more efficient than commercially available non-viral vectors such as PEI and LIPOFECTAMINE® 2000 based on luciferase expression assay while exhibiting minimal toxicity at doses of up to 0.5 mg/ml toxicity compared to these commercially available non-viral vectors. Preferably, the terpolymer is non-toxic at concentrations suitable for both in vitro and in vivo transfection of nucleic acids. For example, in some embodiments, the terpolymers cause less non-specific cell death compared to other approaches of cell transfection. A preferred terpolymer is ω-pentadecalactone-diethyl sebacate-N-methyldiethanolamine terpolymer containing 20% PDL (also referred to as terpolymer III-20% PDL).

Polymers such as PEG-block containing polymers can be used to prepare micelles. The average micelle size is typically in the range from about 100 to about 500 nm, preferably from about 100 to about 400 nm, more preferably from about 100 to about 300 nm, more preferably from about 150 to about 200 nm, most preferably from about 160 to about 190 nm, which were stable at physiological pH of 7.4 in the presence of serum proteins. The copolymers possess high blood compatibility and exhibit minimal activity to induce hemolysis and agglutination.

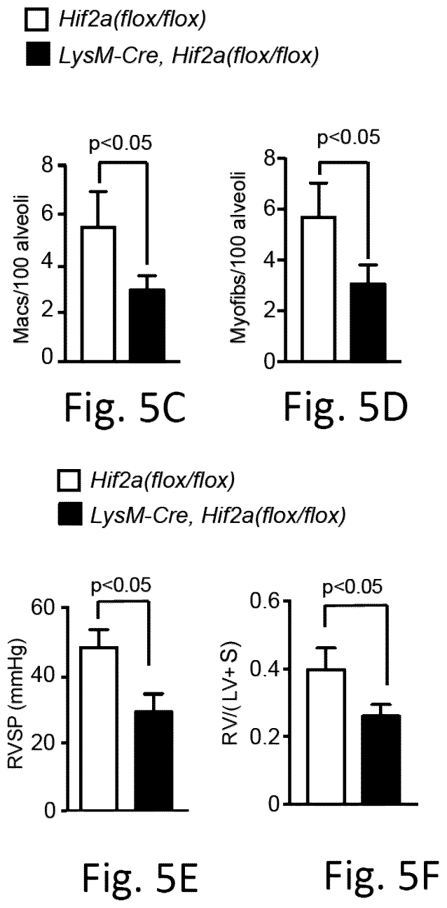

The size and zeta potential of the micelles were found to change significantly when the pH of the aqueous medium accommodating the micelles was varied. For example, the trends in the size-pH and zeta-pH curves are remarkably similar for the micelles of the three PEG2K-PPMS copolymers with different PDL contents (11%, 30%, and 51%). It is evident that the average size of the micelle samples gradually increases upon decreasing the medium pH from 7.4 to 5.0, and then remains nearly constant when the pH value is below 5.0. This pH-responsive behavior observed for the micelles is expected upon decreasing the pH from 7.4 to 5.0, the PPMS cores of the micelles become protonated and more hydrophilic, thus absorbing more water molecules from the aqueous medium to cause swelling of the micelles. The micelle cores are already fully protonated at pH of 5.0, and as a result, the sizes of the micelles remain fairly constant with further decreasing of the pH from 5.0. The effects of the PDL content in the PEG2K-PPMS copolymers on the magnitude of the micelle size change between 7.4 and 5.0 pH values are also notable. With decreasing PDL content and increasing tertiary amino group content in the copolymer, the capacity of the micelle cores to absorb protons and water molecules is expected to increase. Thus, upon decreasing pH from 7.4 to 5.0, the change in average micelle size was more significant for PEG2K-PPMS-11% PDL (from 200 nm to 234 nm) as compared to PEG2K-PPMS-30% PDL (from 184 nm to 214 nm) and PEG2K-PPMS-51% PDL (from 163 nm to 182 nm) (FIG. 5A).

The zeta potential of the micelles in aqueous medium also exhibits substantial pH-dependence. At physiological and alkaline pH (7.4 to 8.5), the surface charges of blank PEG2K-PPMS copolymer micelles were negative, which changed to positive when the pH of the medium decreased to acidic range (4.0-6.0). For example, the micelles of PEG2K-PPMS-11% PDL, PEG2K-PPMS-30% PDL, and PEG2K-PPMS-51% PDL possessed zeta potential values of −5.8, −7.1, −5.1 mV, respectively, at pH of 7.4, which turned to +7.6, +5.8, +4.0 mV, correspondingly, at a lower pH of 5.0. On the basis of the above discussions, this surface charge dependence on pH is attributable to the protonation or deprotonation of the PPMS cores of the micelles at different medium pH. At an alkaline pH (7.4-8.5), most of the amino groups in the micelles presumably are not protonated, and the micelle particles remain negatively charged due to the absorption of HPO42- and/or H2PO4-anions in PBS by the micelles. In particular, at pH of 8.5, the zeta-potential values were −8.1 mV, −7.9 mV, −9.0 mV for PEG2K-PPMS-11% PDL, PEG2K-PPMS-30% PDL, and PEG2K-PPMS-51% PDL, respectively. Upon decreasing pH from 7.4 to 5.0, the tertiary amino moieties in the micelle PPMS cores become mostly protonated, turning the micelles to positively charged particles. Consistently, among the three micelle samples, PEG2K-PPMS-11% PDL micelles with the largest capacity to absorb protons displayed the highest zeta potential values at pH of 4.0-5.0, whereas PEG2K-PPMS-51% PDL micelles with the smallest protonation capacity showed the lowest zeta potentials. The observed micelle surface charge responses to the medium pH are highly desirable since the negative surface charge of the micelles at physiological pH can alleviate the interaction of the micelles with serum protein in the blood and prolong their in vivo circulation time. On the other hand, the reverse to positive surface charge at the tumor extracellular pH of approximately 6.5 could enhance the uptake of these micelles by target tumor cells.

The surface charge of the particles/micelles were slightly negative in PBS solution (0.01M, pH=7.4), which are beneficial for in vivo drug delivery applications of the micelles. It is known that nanoparticles with nearly neutral surface charge (zeta potential between-10 and +10 mV) can decrease their uptake by the reticuloendothelial system (RES) and prolong their circulation time in the blood. The negative surface charges of the micelles could result from the absorption of HPO₄²⁻ and/or H₂PO₄⁻ anions in PBS by the micelle particles via hydrogen bonding interactions between the anions and the ether groups of PEG shells or the amino groups of PPMS cores. For amphiphilic block copolymer micelles, it is anticipated that hydrophilic chain segments (e.g., PEG) in the outer shell of the micelles can shield the charges in the micelle core with the long chain blocks being more effective in reducing zeta potential than the short chain blocks. Thus, significantly lower zeta potential values were observed for PEG5K-PPMS copolymer micelles as compared to PEG2K-PPMS copolymer micelles.

The copolymer micelles are pH-responsive: decreasing the medium pH from 7.4 to 5.0, the sizes of the micelles significantly increased micelle size while the micelle surface charges reversed from negative charges to positive charges. Correspondingly, DTX-encapsulated copolymer micelles showed gradual sustained drug release at pH of 7.4, but remarkably accelerated DTX release at acidic pH of 5.0. This phenomenon can be exploited to improve release of agents at tumor site, since it is known that the tumor microenvironment is typically weakly acidic (e.g., 5.7-7.0) as the result of lactic acid accumulation due to poor oxygen perfusion. In contrast, the extracellular pH of the normal tissue and blood is slightly basic (pH of 7.2-7.4). Thus, enhanced drug delivery efficiency is anticipated for anticancer drug-loaded micelles that are pH-responsive and can be triggered by acidic pH to accelerate the drug release. Furthermore, even more acidic conditions (pH=4.0-6.0) are encountered in endosomes and lysosomes after uptake of the micelles by tumor cells via endocytosis pathways, which may further increase the cytotoxicity of the drug-encapsulated micelles.

### F. Therapeutic, Prophylactic and Diagnostic Agents

The polymers can be used to encapsulate, be mixed with, or be ionically or covalently coupled to any of a variety of therapeutic, prophylactic or diagnostic agents. A wide variety of biologically active materials can be encapsulated or incorporated.

Compounds with a wide range of molecular weight can be encapsulated, for example, between 100 and 500,000 grams or more per mole. In some forms, the agent to be encapsulated and delivered can be a small molecule agent (i.e., non-polymeric agent having a molecular weight less than 2,000, 1500, 1,000, 750, or 500 Dalton) or a macromolecule (e.g., an oligomer or polymer) such as proteins, peptides, nucleic acids, etc. Suitable small molecule active agents include organic, inorganic, and/or organometallic compounds.

Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Examples of suitable materials include proteins such as antibodies, receptor ligands, and enzymes, peptides such as adhesion peptides, saccharides and polysaccharides, synthetic organic or inorganic drugs, and nucleic acids.

Preferred drugs for delivery are those specific for treatment of pulmonary disease or disorder, especially PH. For PH, most drugs are vasodilators meaning that lead to smooth muscle cell relaxation (e.g., endothelin antagonists, prostacyclin analogues, phosphodiesterase inhibitors) which does not make sense to me to use these in a strategy that targets lung macrophages. Drugs that target immune system in PH seem under-utilized. For COPD, similarly inhaled bronchodilators lead to airway SMC relaxation, although one could use corticosteroids are relevant.

Since the results show a surprising selectivity of delivery to, and uptake by, pulmonary immune cells, this delivery system is particularly well suited for local delivery to the lung, especially of antivirals such as those involved in treatment of viral diseases such as COVID-19, diseases such as lung fibrosis, and lung cancer. It also has clear benefits for the delivery of immunomodulators for treatment of chronic obstructive pulmonary disease (COPD).

Exemplary therapeutic agents that can be incorporated into the particles include, but are not limited to, immunomodulatory agents, antiinfectives (including antiviral or antibiotic agents), chemotherapeutic agents, monoclonal antibodies or fragments or humanized versions thereof, enzymes, growth factors, growth inhibitors, hormones, hormone antagonists, and nucleic acid molecules.

Immunomodulatory agents include antiinflammatories, ligands that bind to Toll-Like Receptors to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), and agents that promote uptake of the particles into cells (including dendritic cells and other antigen-presenting cells. Exemplary immunomodulatory agents include cytokines, xanthines, interleukins, interferons, oligodeoxynucleotides, glucans, growth factors (e.g., TNF, CSF, GM-CSF and G-CSF), hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

Oligonucleotide drugs (include DNA, RNAs, antisense, aptamers, small interfering RNAs, ribozymes, external guide sequences for ribonuclease P, and triplex forming agents.

Representative chemotherapeutic agents include alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®).

Examples of immunological adjuvants that can be associated with the particles include, but are not limited to, TLR ligands, C-Type Lectin Receptor ligands, NOD-Like Receptor ligands, RLR ligands, and RAGE ligands. TLR ligands can include lipopolysaccharide (LPS) and derivatives thereof, as well as lipid A and derivatives there of including, but not limited to, monophosphoryl lipid A (MPL), glycopyranosyl lipid A, PET-lipid A, and 3-O-desacyl-4′-monophosphoryl lipid A.

The particles may also include antigens and/or adjuvants (i.e., molecules enhancing an immune response). Peptide, protein, and DNA based vaccines may be used to induce immunity to various diseases or conditions. Cell-mediated immunity is needed to detect and destroy virus-infected cells. Most traditional vaccines (e.g. protein-based vaccines) can only induce humoral immunity. DNA-based vaccine represents a unique means to vaccinate against a virus or parasite because a DNA based vaccine can induce both humoral and cell-mediated immunity. DNA vaccines consist of two major components, DNA carriers (or delivery vehicles) and DNAs encoding antigens. DNA carriers protect DNA from degradation, and can facilitate DNA entry to specific tissues or cells and expression at an efficient level.

Representative diagnostic agents include agents detectable by x-ray, fluorescence, magnetic resonance imaging, radioactivity, ultrasound, computer tomagraphy (CT) and positron emission tomagraphy (PET). Ultrasound contrast agents are typically a gas such as air, oxygen or perfluorocarbons. Exemplary diagnostic agents include paramagnetic molecules, fluorescent compounds, magnetic molecules, and radionuclides, and x-ray imaging agents.

In some embodiments, particles produced using the methods described herein contain less than 80%, less than 75%, less than 70%, less than 60%, less than 50% by weight, less than 40% by weight, less than 30% by weight, less than 20% by weight, less than 15% by weight, less than 10% by weight, less than 5% by weight, less than 1% by weight, less than 0.5% by weight, or less than 0.1% by weight of the agent. In some embodiments, the agent may be a mixture of pharmaceutically active agents. The percent loading is dependent on a variety of factors, including the agent to be encapsulated, the polymer used to prepare the particles, and the method used to prepare the particles.

### Polynucleotides

The polymeric particles can be used to transfect cells with nucleic acids. The polynucleotide can encode one or more proteins, functional nucleic acids, or combinations thereof. The polynucleotide can be monocistronic or polycistronic. In some embodiments, the polynucleotide is multigenic.

In some embodiments, the polynucleotide is transfected into the cell and remains extrachromosomal. In some embodiments, the polynucleotide is introduced into a host cell and is integrated into the host cell's genome.

In some embodiments, the polynucleotide is incorporated into or part of a vector. Methods to construct expression vectors containing genetic sequences and appropriate transcriptional and translational control elements are well known in the art. These methods include in vitro recombinant DNA techniques, synthetic techniques, and in vivo genetic recombination. Expression vectors generally contain regulatory sequences and necessary elements for the translation and/or transcription of the inserted coding sequence, which can be, for example, the polynucleotide of interest. The coding sequence can be operably linked to a promoter and/or enhancer to help control the expression of the desired gene product. Promoters used in biotechnology are of different types according to the intended type of control of gene expression. They can be generally divided into constitutive promoters, tissue-specific or development-stage-specific promoters, inducible promoters, and synthetic promoters.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

Specific initiation signals may also be required for efficient translation of the compositions. These signals include the ATG initiation codon and adjacent sequences. Exogenous translational control signals, including the ATG initiation codon, may additionally need to be provided. In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

In preferred embodiments, the polynucleotide cargo is an RNA, such as an mRNA. The mRNA can encode a polypeptide of interest.

In some embodiments, the mRNA has a cap on the 5′ end and/or a 3′ poly(A) tail which can modulateribosome binding, initiation of translation and stability mRNA in the cell.

The polynucleotide can encode one or more polypeptides of interest.

In some embodiments, the polynucleotide supplements or replaces a polynucleotide that is defective in the organism.

In some embodiments, the polynucleotide includes a selectable marker, for example, a selectable marker that is effective in a eukaryotic cell, such as a drug resistance selection marker. In some embodiments, the polynucleotide includes a reporter gene.

The polynucleotide can be, or can encode a functional nucleic acid. Functional nucleic acids are nucleic acid molecules that have a specific function, such as binding a target molecule or catalyzing a specific reaction. Functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation.

Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA: EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors.

The polynucleotide can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds.

The polynucleotide can be composed of nucleotide analogs that have been chemically modified to improve stability, half-life, or specificity or affinity for a target sequence, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge. Modifications should not prevent, and preferably enhance, the ability of the oligonucleotides to enter a cell and carry out a function such inhibition of gene expression as discussed above.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

Efficiency of polynucleotide delivery using the polymers can be affected by the positive charges on the polyplex surface. For example, a zeta potential of the polyplex of +8.9 mV can attract and bind with negatively charged plasma proteins in the blood during circulation and lead to rapid clearance by the reticuloendothelial system (RES). Efficiency can also be affected by instability of the polyplex nanoparticles. For example, as discussed in the Examples below, polyplex particles incubated in NaAc buffer solution containing 10% serum nearly doubled in size within 15 minutes and increased by over 10-fold after 75 minutes. As a result of this increase in size, enlarged polyplexes might be cleared from the circulation by uptake in the liver. Therefore, in some embodiments the polyplexes are treated or coated to improve polynucleotide delivery efficiency. In some embodiments, the coating improves cell specific targeting of the polyplex, improves the stability (i.e., stabilizes the size of the polyplex in vivo), increases the half-life of the polyplex in vivo (i.e., in systemic circulation), or combinations thereof compared to a control. In some embodiments, the control is a polyplex without a coating.

An exemplary polyplex coating for targeting tumor cells is polyE-mRGD. As used herein, polyE-mRGD refers to a synthetic peptide containing three segments: a first segment including a polyglutamic acid (polyE) stretch, which is negatively charged at physiological pH and, therefore, capable of electrostatic binding to the positively charged surface of the polyplexes; a second segment including a neutral polyglycine stretch, which serves as a neutral linker; and a third segment that includes a RGD sequence that binds the tumor endothelium through the interaction of RGD with αᵥβ₃ and αᵥβ₅.

Polynucleotide delivery efficiency of the polyplexes can be improved by coating the particles with an agent that is negatively charged at physiological pH. Preferably, the negatively charged agent is capable of electrostatic binding to the positively charged surface of the polyplexes. The negatively charged agent can neutralize the charge of the polyplex, or reverse the charge of the polyplex. Therefore, in some embodiments, the negatively charged agent imparts a net negative charge to the polyplex.

In some embodiments, the negatively charged agent is a negatively charged polypeptide. For example, the polypeptide can include aspartic acids, glutamic acids, or a combination therefore, such that the overall charge of the polypeptide is a negative at neutral pH. Increasing the negative charge on the surface of the particle can reduce or prevent the negative interactions described above, wherein more positively charged particles attract and bind negatively charged plasma proteins in the blood during circulation and lead to rapid clearance by the reticuloendothelial system (RES). In some embodiments, the zeta potential of the particles is from about −15 mV to about 10 mV, preferably from about −15 mV to about 8 mV, more preferably from about −10 mV to about 8 mV, more preferably from about −8 mV to about 8 mV. The zeta potential can be more negative or more positive than the ranges above provided the particles are stable (i.e., don't aggregate, etc.) and not readily cleared from the blood stream The zeta potential can be manipulated by coating or functionalizing the particle surface with one or more moieties which varies the surface charge. Alternatively, the monomers themselves can be functionalized and/or additional monomers can be introduced into the polymer, which vary the surface charge.

Resistance to aggregation can be important because maintaining a small particle size limits clearance by the liver and maintains transfection ability of polyplex particles into target cells. Therefore, in preferred embodiments, the polyplexes are resistant to aggregation. Preferably, polyplexes with or without coating are between about 1 nm and 1000 nm in radius, more preferably between about 1 nm and about 500 nm in radius, most preferably between about 15 nm and about 250 nm in radius. For example, in some embodiments, coated polyplexes loaded with polynucleotide are between about 150 nm and 275 nm in radius.

The ratio of polynucleotide weight to polymer weight (polynucletide:polymer), the content and quantity of polyplex coating, or a combination thereof can be used to adjust the size of the polyplexes.

### G. Formulations

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, desintegrators, fillers, and coating compositions.

For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

Preferred formulations for pulmonary delivery are pharmaceutically acceptable carriers for administration by aerosol, inhaler, dry powder, intubation and instillation.

### III. Methods of Preparing Particles or Polyplexes

Particles can be prepared using a variety of techniques known in the art. The technique to be used can depend on a variety of factors including the polymer used to form the nanoparticles, the desired size range of the resulting particles, and suitability for the material to be encapsulated.

Methods known in the art that can be used to prepare nanoparticles include, but are not limited to, polyelectrolyte condensation (see Suk et al., Biomaterials, 27, 5143-5150 (2006)); single and double emulsion; nanoparticle molding, and electrostatic self-assembly (e.g., polyethylene imine-DNA or liposomes).

In one embodiment, the loaded particles are prepared by combining a solution of the polymer, typically in an organic solvent, with the polynucleotide of interest. The polymer solution is prepared by dissolving or suspending the polymer in a solvent. The solvent should be selected so that it does not adversely effect (e.g., destabilize or degrade) the nucleic acid to be encapsulated. Suitable solvents include, but are not limited to DMSO and methylene chloride. The concentration of the polymer in the solvent can be varied as needed. In some embodiments, the concentration is for example 25 mg/ml. The polymer solution can also be diluted in a buffer, for example, sodium acetate buffer.

Next, the polymer solution is mixed with the agent to be encapsulated, such as a polynucleotide. The agent can be dissolved in a solvent to form a solution before combining it with the polymer solution. In some embodiments, the agent is dissolved in a physiological buffer before combining it with the polymer solution. The ratio of polymer solution volume to agent solution volume can be 1:1. The combination of polymer and agent are typically incubated for a few minutes to form particles before using the solution for its desired purpose, such as transfection. For example, a polymer/polynucleotide solution can be incubated for 2, 5, 10, or more than 10 minutes before using the solution for transfection. The incubation can be at room temperature.

In some embodiments, the particles are also incubated with a solution containing a coating agent prior to use. The particle solution can be incubated with the coating agent for 2, 5, 10, or more than 10 minutes before using the polyplexes for transfection. The incubation can be at room temperature.

In some embodiments, if the agent is a polynucleotide, the polynucleotide is first complexed to a polycation before mixing with polymer. Complexation can be achieved by mixing the polynucleotides and polycations at an appropriate molar ratio. When a polyamine is used as the polycation species, it is useful to determine the molar ratio of the polyamine nitrogen to the polynucleotide phosphate (N/P ratio). In a preferred embodiment, inhibitory RNAs and polyamines are mixed together to form a complex at an N/P ratio of between approximately 1:1 to 1:25, preferably between about 8:1 to 15:1. The volume of polyamine solution required to achieve particular molar ratios can be determined according to the following formula:

$${V}_{NH 2} = \frac{{C}_{inhRNA , final} \times {M}_{w , inhRNA} / {C}_{inhRNA , fina1} \times {M}_{w , P} \times {Φ}_{N : P} \times Φ {V}_{final}}{{C}_{NH 2} / {M}_{w , NH 2}}$$

where M_{w,inhRNA}=molecular weight of inhibitory RNA, M_{w,P}=molecular weight of phosphate groups of inhibitory RNA, Φ_{N:P}=N:P ratio (molar ratio of nitrogens from polyamine to the ratio of phosphates from the inhibitory RNA), C_{NH2}, stock=concentration of polyamine stock solution, and M_{w,NH2}=molecular weight per nitrogen of polyamine. Methods of mixing polynucleotides with polycations to condense the polynucleotide are known in the art. See for example U.S. Published Application No. 2011/0008451.

The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values. Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In some embodiments, the polycation is a polyamine. Polyamines are compounds having two or more primary amine groups. Suitable naturally occurring polyamines include, but are not limited to, spermine, spermidine, cadaverine and putrescine. In a preferred embodiment, the polyamine is spermidine.

In another embodiment, the polycation is a cyclic polyamine. Cyclic polyamines are known in the art and are described, for example, in U.S. Pat. No. 5,698,546, WO 1993/012096 and WO 2002/010142. Exemplary cyclic polyamines include, but are not limited to, cyclen.

Spermine and spermidine are derivatives of putrescine (1,4-diaminobutane) which is produced from L-ornithine by action of ODC (ornithine decarboxylase). L-ornithine is the product of L-arginine degradation by arginase. Spermidine is a triamine structure that is produced by spermidine synthase (SpdS) which catalyzes monoalkylation of putrescine (1,4-diaminobutane) with decarboxylated S-adenosylmethionine (dcAdoMet) 3-aminopropyl donor. The formal alkylation of both amino groups of putrescine with the 3-aminopropyl donor yields the symmetrical tetraamine spermine. The biosynthesis of spermine proceeds to spermidine by the effect of spermine synthase (SpmS) in the presence of dcAdoMet. The 3-aminopropyl donor (dcAdoMet) is derived from S-adenosylmethionine by sequential transformation of L-methionine by methionine adenosyltransferase followed by decarboxylation by AdoMetDC (S-adenosylmethionine decarboxylase). Hence, putrescine, spermidine and spermine are metabolites derived from the amino acids L-arginine (L-ornithine, putrescine) and L-methionine (dcAdoMet, aminopropyl donor).

### IV. Methods of Using the Particles/Micelles

The particles can be used to deliver an effective amount of one or more therapeutic, diagnostic, and/or prophylactic agents to a patient in need of such treatment. The amount of agent to be administered can be readily determine by the prescribing physician and is dependent on the age and weight of the patient and the disease or disorder to be treated.

The compositions are administered to the lungs of a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

### EXAMPLES

The present invention will be further understood by reference to the following non-limiting examples showing how one can selectively treat one or more symptoms of pulmonary hypertension by selective targeting of a platelet-derived growth factor inhibitor using PACE nanoparticles.

### Pathological Characteristics of Pulmonary Hypertension (“PH”):

distal pulmonary arteriole muscularization
elevated pulmonary artery blood pressure
right ventricular hypertrophy (RVH)

Platelet-derived growth factor (PDGF)—B from endothelial cells is important for PH pathogenesis, but the role of lung macrophages and macrophage-derived PDGF-B in PH is not well delineated.

The following studies demonstrate that lung macrophage-derived PDGF-β plays a key role in pathological SMC expansion in PH, and that inhibitors of PDGF-β can be selectively delivered to pulmonary macrophages and monocytes for treatment thereof.

### Example 1: Alveolar and Parenchymal Lung Macrophages Accumulate in Hypoxia and Their Depletion Attenuates Distal Muscularization and PH

A model of PH in which wild type or transgenic mice were exposed to hypoxia for up to 21 days. Measurements and analysis were conducted on lung tissue, BALF cells and heart. Furthermore, studies were conducted on fresh whole blood from human patients in which primary monocytes were isolated and differentiated into macrophages and the RNA content analyzed in these cells as well as the effects of the conditioned medium from such cultures on SMCs migration and proliferation.

### Methods and Materials

### Animal Studies

Mice were obtained from the Jackson Laboratory. C57BL/6 mice were used for wild type studies, and mice carrying LysM-Cre (Clausen Transgenic Res. 1999; 8(4):265-77; Cowburn. Proc Natl Acad Sci USA. 2016; 113(31):8801-6), ROSA26R^{(mTmG/mTmG)} (Muzumdar M D, Tasic B, Miyamichi K, Li L, and Luo L. A global double-fluorescent Cre reporter mouse. Genesis. 2007; 45(9):593-605), PDGF-β^{(flox/flox)} (Enge M, et al. EMBO J. 2002; 21(16):4307-16), Vhl^{(flox/flox)} (Haase Proc Natl Acad Sci USA. 2001; 98(4):1583-8), Hif1α^{(flox/flox)} (Ryan. Cancer Res. 2000; 60(15):4010-5) or Hif2α^{(flox/flox)} (Gruber, Proc Natl Acad Sci USA. 2007; 104(7):2301-6). Male and female mice aged 10⁻¹⁶ weeks and sex and age-matched controls were used.

### Hypoxia Exposure and Hemodynamic Measurements

Mice were placed for up to 21 days in a hypoxia (10% FiO₂) chamber equipped with a controller and oxygen sensor (BioSpherix®). Following hypoxia treatment, RVSP was measured. Mice were then euthanized by isoflurane inhalation, and in addition to lung harvesting, hearts were collected to determine the Fulton index, which is the weight ratio of the RV to the sum of the LV and septum(S) ((Sheikh Cell Rep. 2014; 6(5):809-17). The technician conducting hemodynamic measurements was blinded as to the treatment group and genotype of mice.

### Bronchoalveolar Lavage Fluid and Lung Harvesting

Following euthanasia, PBS was perfused through the RV into the lungs. When the whole lung was analyzed, both the right and left lungs were harvested directly after perfusion. For BALF collection, 1 ml PBS was injected through the trachea into alveoli and then aspirated from the trachea. This procedure was repeated once, and the collected BALF was pooled. The BALF was centrifuged at 830 g (GS-6R centrifuge, Beckman Coulter) for 10 min at 4° C., and the cell pellet was collected. For FACS experiments on the residual lung, following BALF removal, the right main stem bronchus was ligated, and the right lung was removed. For immunohistochemistry, the left lung was inflated with 2% low-melt agarose and placed in ice-cold PBS. When the agarose solidified, the left lung was immersed in Dent's fixative (4:1 methanol: DMSO) at 4° C. overnight and the next day was washed and stored in 100% methanol at −80° C.

### Nanoparticle Formulation and Administration

Nanoparticles were orotracheally administered to wild type mice. Clodronate- or PDGF-β siRNA-loaded nanoparticles were administered at the onset of hypoxia and every three days thereafter for up 21 days of hypoxia. Mice receiving nanoparticles loaded with the dye DiD were maintained in normoxia for 6 h and then euthanized. For phagocyte depletion, 50 μL of liposomes loaded with 0.25 mg clodronate or PBS and dissolved in PBS (Liposoma Research) were injected. For nanoparticle uptake assessment or PDGF-β knockdown, PACE nanoparticles composed of acid-ended (poly(pentadecalactone-co-n-methyldiethanolamineco-sebacate) with 50% lactone (PPMS-50COOH) were formulated using a modified single emulsion or double emulsion solvent evaporation technique (Kauffman Biomacromolecules. 2018; 19(9):3861-73). Briefly, in formulation of dye-loaded nanoparticles (˜200 or ˜400 nm in diameter), 0.2 wt % of DiD (ThermoFisher) to polymer was used. DMSO (10 μL of 10 mg/mL solution) was dissolved into 50 mg of polymer immediately prior to single emulsion formulation. For PDGF-β siRNA and scrambled (Scr) RNA-loaded nanoparticles, the nucleic acid cargo (Dharmacon, 50 nM) was dissolved in sodium acetate buffer (25 mM, pH 5.8) before proceeding to the double emulsion method. Parameters of nanoparticles (stratified by siPDGF-β or Scr loading) were assayed, including hydrodynamic diameter (404±8 or 386±7 nm), size distribution (PDI; 0.218±0.004 or 0.238±0.007) and zeta potential (9.4±0.3 or 10.8±0.5 mV) using dynamic light scattering (Zetasizer Pro, Malvern Panalytical) and siRNA loading efficiency (69.6±1.2 or 64.3±0.5%) using QuantIT RiboGreen assay (ThermoFisher). Nanoparticles (0.2 mg) were suspended in 50 μL PBS and administered to mice. To confirm uptake of nanoparticles by macrophages in culture, BALF cell pellet was resuspended in murine cell culture medium (RPMI [Thermo Scientific], 10% fetal bovine serum [FBS; Invitrogen], 5% penicillin/streptomycin [Life Technologies]) and incubated with 0.25 mg/ml DiD-loaded nanoparticles for 6 h at 37° C.

### Immunohistochemistry

For immunohistochemical analysis, left lungs stored in 100% methanol were subjected to peroxidase deactivation by incubation in 5% H₂O₂/methanol for 15 min at RT and then sequentially rehydrated in 75%, 50% and 25% and 0% methanol in PBS. A vibratome was used to cut the rehydrated lung into 150 μm thick sections, which were incubated in IHC blocking buffer (5% goat serum in 0.5% Triton X-100/PBS [PBS-T]) at 4° C. overnight and then stained with primary antibodies in IHC blocking buffer for 3 days at 4° C. Subsequently, sections were washed three times in PBS-T, incubated in secondary antibodies in IHC blocking buffer overnight at 4° C., washed five times in PBS-T, mounted on slides with Dako mounting medium and stored at 4° C. Primary antibodies used were rat anti-MECA-32 (1:15, Developmental Studies Hybridoma Bank [DSHB]), rat anti-CD31-FITC (1:250, BD Biosciences), mouse anti-CD64-APC (1:250, Biolegend), rat anti-CD68-APC (1:50, Miltenyi Biotec) and mouse anti-SMA-Cy3 clone 1A4 (1:250, Sigma). Secondary antibody used was Alexa 488 anti-rat (1:250, Invitrogen). Nuclei were stained with DAPI (1:500).

### Imaging

Images of the stained sections were acquired using confocal microscopes (PerkinElmer Ultra View VOX spinning disc or Leica SP8 point scanning). Adobe Photoshop was used to process images. For analysis of distal muscularization, we focused on two specific arteriole beds in the left lung previously described and denoted as L.L1.A1.L1 and L.L1.A1.M1 (Sheikh 2014; Sheikh 2015). Their nomenclature derives from the nearest airways that have a stereotyped branching pattern in the adult mouse (Sheikh et al Cell Rep. 2014; 6(5):809-17, Metzger. Nature. 2008; 453(7196):745-50). Based on their diameter and branching pattern, pulmonary arterioles are classified as proximal (P; >75 mm diameter), middle (M; 25 to 75 mm), and distal (D; <25 mm) and the names L, left main bronchus; L1, L2, L3, lateral branches; M1, M2 medial branches; A1, A2 anterior branches.

### Human Studies

All procedures involving human subjects were approved by the Institutional Review Board of Yale University (IRB #1307012431 and #1005006865), and we complied with all relevant ethical regulations. Written informed consent was obtained from all participants prior to inclusion in the study.

### Human Monocyte Isolation and Differentiation to Macrophages

Fresh whole blood from IPAH and SSc-PAH patients of the Pulmonary Vascular Disease clinic at Yale University School of Medicine and healthy controls were provided to the Greif lab as de-identified samples. Monocytes were isolated and differentiated into macrophages based on methods described previously (Bennett J Exp Med. 1966; 123(1):145-60; Karlsson, et al. Exp Hematol. 2008; 36(9):1167-75). In brief, fresh whole blood was diluted 3-fold in HBSS, loaded on a Ficoll-Histopaque column (Fisher Scientific) and centrifuged for 30 min at 830 g. The peripheral blood mononuclear phase was aspirated, diluted 3-fold in HBSS and centrifuged for 10 min at 830 g. To ensure platelet removal, the pellet was resuspended in 3 ml HBSS and centrifuged for an additional 10 min at 830 g. The pellet was then resuspended in RPMI with 10% FBS, and cells were allowed to adhere to a plastic cell culture dish for 1 h at 37° C. Monocytes preferentially adhere to plastic (37) (Fig. S6A, B). Floating cells were discarded, and adherent cells were washed with PBS and either incubated with 5 mM EDTA in PBS for 10 min and collected for staining and flow cytometry or cultured in macrophage differentiation medium (ImmunoCult™-SF macrophage medium and 1 ng/ml macrophage colony-stimulating factor [both from StemCell Technologies]). The medium was replaced by fresh macrophage differentiation medium on the fourth day. On day 6, the medium was changed to ImmunoCult™-SF macrophage medium, and 12 h later, conditioned medium was collected, and cells were harvested. For hypoxia studies, macrophages derived from monocytes of healthy donors were exposed to either normoxia or 3% O₂ for 12 h in RPMI supplemented with 1% FBS and 5% penicillin-streptomycin.

hPASMC Culture and Proliferation Assay

hPASMCs (American Type Culture Collection) were cultured up to passage 6 in M199 medium supplemented with 10% FBS, 1% penicillin/streptomycin, 2 ng/ml fibroblast growth factor (Promega), 3 ng/ml epidermal growth factor (Promega). Proliferation was assessed as previously described with minor modifications (Dave J. Dev Cell. 2018; 44(6):665-78 e6). hPASMCs were trypsinized and cultured overnight on culture slides (BD Falcon) pre-coated with fibronectin (10 μg/mL in PBS). On the next day, the cells were washed with PBS and serum starved overnight in M199 supplemented with 0.5% FBS. Cells were then washed in PBS and cultured for 24 h in medium conditioned by human control or patient-derived macrophages that had or had not been pre-treated with 20 μg/ml IgG control or anti-PDGF-B blocking antibody (R&D Systems) for 1 h at 37° C. For the final 10 h of this incubation, 10 μg/ml BrdU (Sigma) was added to the cells. Slides were fixed in 4% paraformaldehyde for 30 min, rinsed in 0.3% Tris, 1.5% glycine in water for 15 min, incubated in 2N HCl for 30 min at 37° C., washed with 0.1 M boric acid and then incubated in 1% FBS in PBS-T for 1 h. hPASMCs were stained with rat anti-BrdU primary antibody (1:100, BioRad) in 1% FBS in PBS-T for 1 h, washed three times in 0.5% Tween 20 in PBS and then incubated with goat anti-rat secondary antibody conjugated to Alexa 488 (1:500, Molecular Probes) and PI (1:500, Sigma) in 1% FBS in PBS-T for 1 h. Finally, slides were washed three times in 0.5% Tween 20 in PBS and mounted on slides using fluorescence mounting medium (Dako). Proliferation was calculated as the percentage of total PI⁺ hPASMCs that were BrdU⁺. For each control or patient, at least 10 fields of view were scored.

### SMC Migration Assay

Cell migration was assessed by the method by Dave Dev Cell. 2018; 44(6):665-78 e6. Briefly, hPASMCs were trypsinized and immediately added to the top of Boyden chamber polycarbonate membranes (Corning Costar, 8 μm pores). The lower compartment of the Boyden chamber contained medium conditioned by human control and patient-derived macrophages that was or was not pre-treated with 20 μg/ml anti-PDGF-B blocking antibody or IgG control for 1 h at 37° C. hPASMCs were allowed to migrate for 8 h towards the lower chamber at which time the membrane was fixed in 4% paraformaldehyde for 30 min, stained with 0.1% Crystal Violet and washed with water. The upper surface of the membrane was scraped with a cotton swab to remove non-migrated cells, and cells on the bottom surface (i.e., migrated cells) were imaged and counted.

### Statistics

All data are presented as mean values±standard deviation. Student's t-test (unpaired, two-tailed) and one-way ANOVA were used to compare means of two groups and multiple groups, respectively (GraphPad Prism software). The statistical significance threshold was set at p≤0.05. All tests assumed normal distribution.

### Results

FIGS. 1A-1 of the results shows that alveolar and residual parenchymal lung macrophages, CD64⁺Ly6G⁻ cells, accumulate in hypoxia.

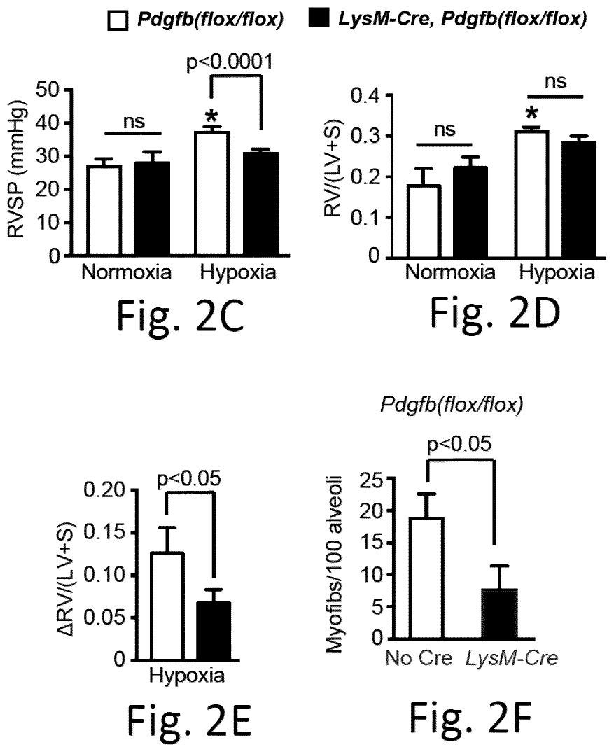

As shown by FIGS. 2A-2B, similarly, there is an increase in PDGF-mRNA which peaked at a level of ˜6 and ˜9-fold increased for alveolar and residual lung macrophages, respectively.

FIGS. 2C-2F demonstrates that macrophage depletion attenuates muscularization, right ventricular systolic pressure as well as right ventricle hypertrophy in hypoxia-conditioned animals.

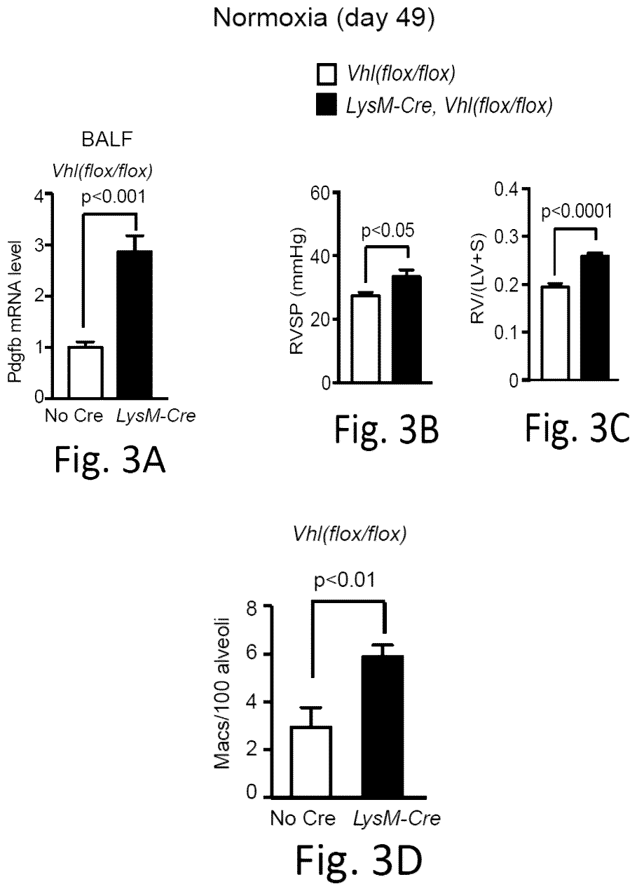

LysM-Cre mice with floxed alleles were used to delete specific genes in myeloid cells. After 21 days of hypoxia, mice with myeloid cells depleted in PDGF-β or the hypoxia-inducible factor 2a are protected against distal arteriole muscularization and PH. As shown by FIGS. 3A-3D, von-Hippel Lindau plays a key role in the degradation of hypoxia inducible factors, and the results indicate that deletion of the von-Hippel Lindau gene in myeloid leads to distal arteriole muscularization and PH in normoxia.

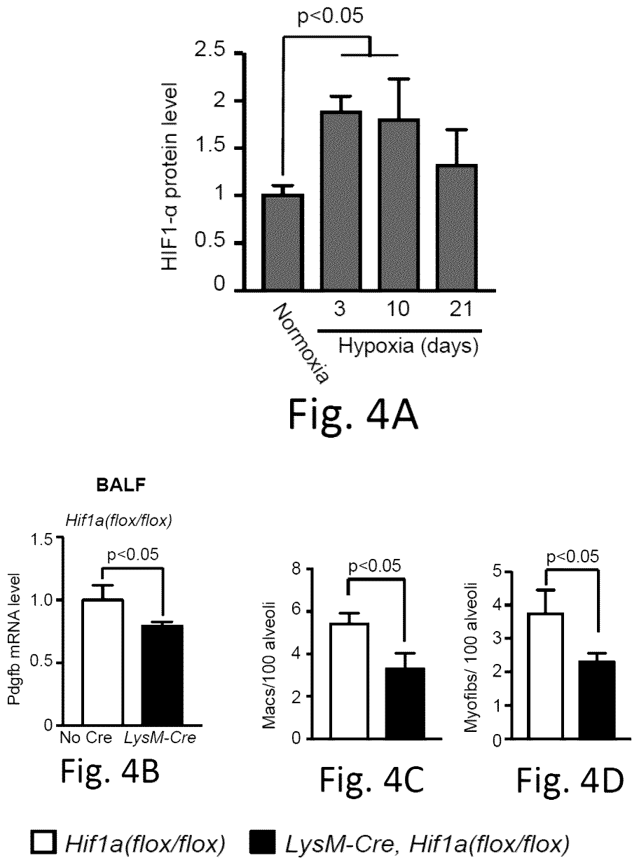

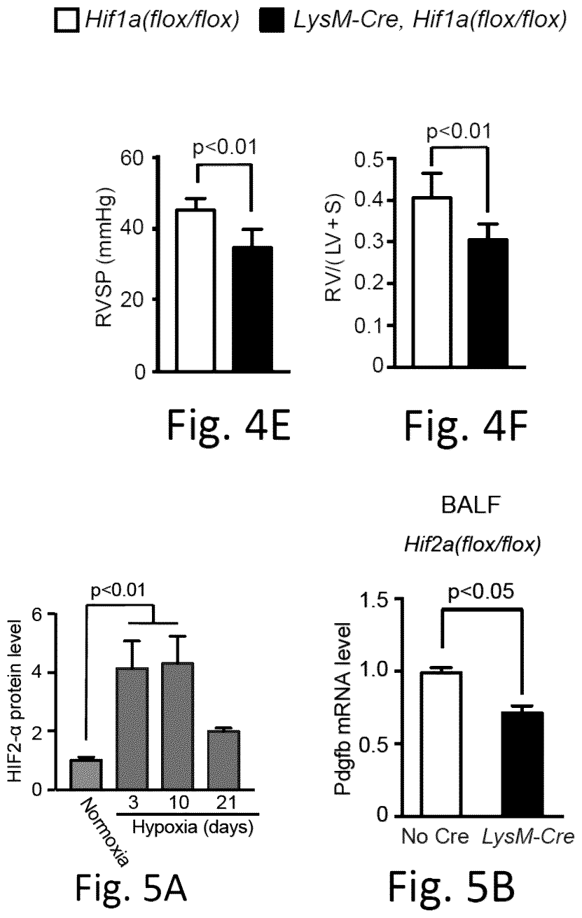

As shown by FIGS. 4A-4F, FIGS. 5A-5F, and FIGS. 7A-7F, the effect of pharmacologically downregulating PDGF-β in lung macrophages by delivering nanoparticles loaded with PDGF-β siRNA was assessed. In bronchoalveolar lung fluid, PDGF-β siRNA reduces PDGF-β levels by 90%. These siPDGF-β nanoparticles attenuate hypoxia-induced distal pulmonary arteriole muscularization, PH and right ventricle hypertrophy.

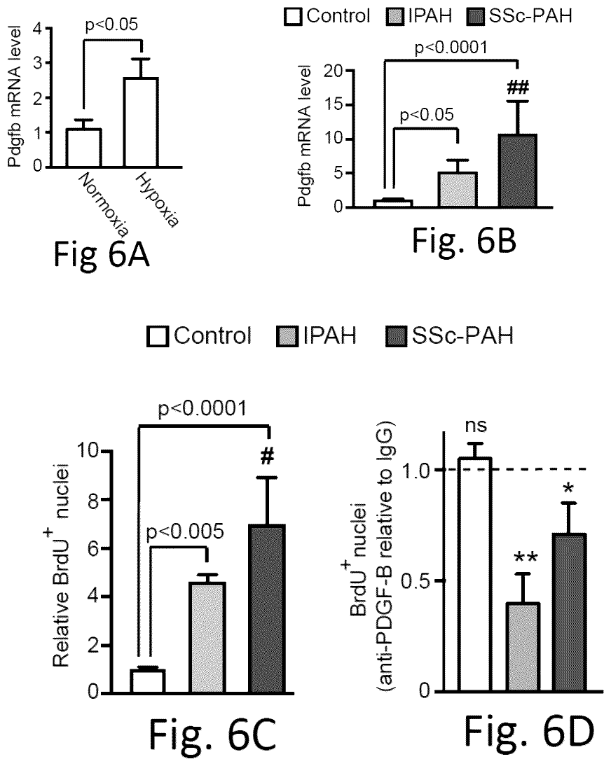

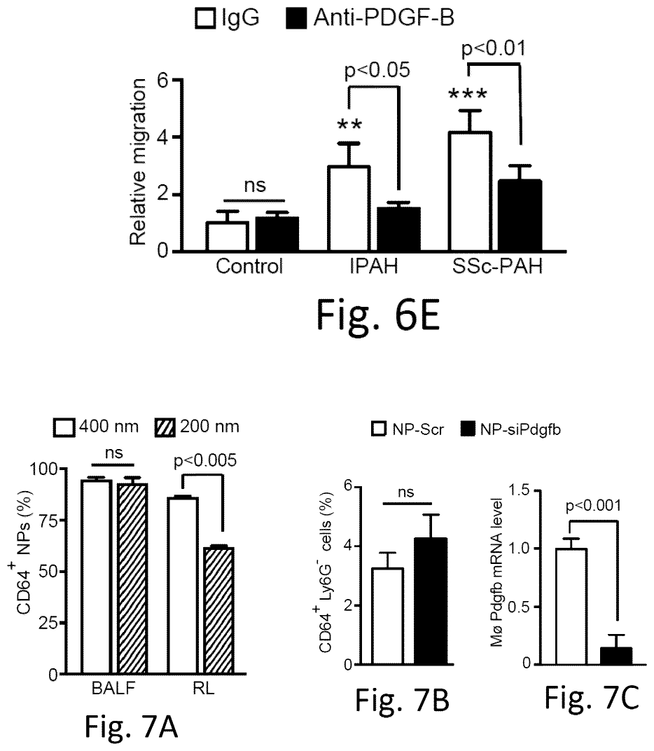

Finally, in FIGS. 6A-6E to assess the clinical relevance of this work, human macrophages and SMCs were studied. Initially, in macrophages from healthy donors, there was a 2.5-fold increase in PDGF-β transcript level with exposure to hypoxia (FIG. 6A-6B). Additionally, PDGF-β levels in macrophages from patients with PH due to an idiopathic etiology or scleroderma were enhanced by 5 and 10-fold, respectively. See FIGS. 6C-6D. Also, medium conditioned by patient macrophages increased SMC proliferation by ˜6-fold. Furthermore, pre-treatment of PH patient conditioned medium with anti-PDGF-B blocking antibody inhibited this SMC proliferation. See FIG. 6E. Similarly, PH patient conditioned medium induced SMC migration by ˜4 fold, and anti-PDGF-B pre-treatment reduced this effect by ˜50%.

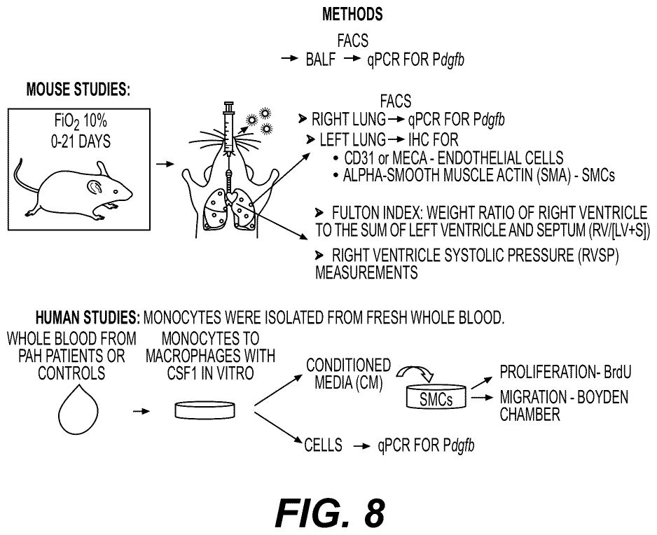

FIG. 8 is a schematic of the summary of the methods used herein for the mouse and human studies.

Taken together, the studies with an experimental model as well as cells isolated from human pulmonary hypertension patients demonstrate that macrophage hypoxia-inducible factor and PDGF-B plays a major role in SMC and right ventricle remodeling and PH. Furthermore, nanoparticle-mediated silencing of PDGF-β in lung macrophages is a therapeutic s

Immunohistochemical analysis of distal muscularization in the investigations herein focused on specific pulmonary arteriole beds adjacent to identified airway branches left bronchus-first lateral secondary branch-first anterior branch-first lateral or first medial branch (L.L1.A1.L1 or L.L1.A1.M1). Under normoxic conditions, distal arterioles in these beds are unmuscularized but undergo a stereotyped process of muscularization with hypoxia exposure (Sheikh Cell Rep. 2014; 6(5):809-17; Sheikh Sci Transl Med. 2015; 7(308):308ra159; Sheikh Cell Rep. 2018; 23(4):1152-65).

In addition to developing distal arteriole muscularization and PH, the lungs of mice exposed to hypoxia accumulate excess macrophages (Amsellem Am J Respir Cell Mol Biol. 2017; 56(5):597-60818, Stenmark Circ Res. 2006; 99(7):675-91; Rabinovitch Annu Rev Pathol. 2007; 2:369-99) (FIG. 1A-C). The time course of lung macrophage accumulation during PH in wild type mice maintained in hypoxia (FiO₂ 10%) was determined for up to 21 days. The pulmonary vasculature was flushed and then using flow cytometry, CD64⁺Ly6G⁻ macrophages were isolated from bronchoalveolar lavage fluid (BALF) and from the residual lung after BALF. The percent of macrophages in BALF gradually increases reaching statistical significance on hypoxia day 21 in comparison to normoxia. In contrast, macrophages from the residual lung are 2.9±0.5-fold increased by hypoxia day 3 and up to 10.8±1.1-fold increased at hypoxia day 21.

The effects of depletion of alveolar and residual macrophages with clodronate on hypoxia-induced distal muscularization and PH was assessed. Liposomes loaded with clodronate or as a control with phosphate buffered saline (PBS) were administered orotracheally to wild type mice at the onset of hypoxia and two times per week during the ensuing 21 days of hypoxia to deplete phagocytes. Mice treated with clodronate had attenuated hypoxia-induced distal muscularization, right ventricular systolic pressure (RVSP; equivalent to pulmonary artery systolic pressure) and RVH as measured by the Fulton index (i.e., weight ratio of the right ventricle [RV] to the sum of the left ventricle [LV] and septum [S]). In comparison to control liposomes, treatment with clodronate-loaded liposomes reduced macrophages by ˜50% in the BALF and ˜65% in the residual lung (FIG. 1E, IF). Under basal conditions, the adult lung has very rare myofibroblasts, but it has been demonstrated that hypoxia induces a marked increase in the number of these cells (Sheikh Cell Rep. 2014; 6(5):809-17, Chen J Appl Physiol (1985). 2006; 100(2):564-71). Depletion of myeloid cells markedly inhibits hypoxia-induced accumulation of alveolar myofibroblasts.

Lung Macrophage PDGF-β is Upregulated with Hypoxia and PDGF-β Deletion in the LysM-Cre Lineage Attenuates PH

Exposure of mice to hypoxia increases PDGF-B levels in the whole lung and in lung ECs specifically (Sheikh 2015; Sheikh 2018); however, not all lung PDGF-B derives from EC. Thus, a time course of PDGF-β expression in CD64⁺Ly6G⁻ macrophages isolated by FACS from the BALF and residual lung of mice exposed to hypoxia for up to 21 days was calculated. PDGF-β mRNA level was measured by qRT-PCR and in comparison to normoxia, was increased within one day of hypoxia and peaked at day 3 at a level of 5.6±0.2 and 9.3±0.2-fold increased for BALF and residual lung, respectively (FIG. 2A, B). To further confirm the upregulation of PDGF-β in monocytes/macrophages, LysM-Cre which marks this population was used. LysM-Cre, ROSA26R^{(mTmG/mTmG)} mice were exposed to hypoxia for 21 days or maintained in normoxia, and then GFP cells were isolated by FACS from whole lung. PDGF-β mRNA level was increased by 2.1±0.4 fold in cells isolated from hypoxic mice. Similarly, GFP cells isolated from BALF of normoxic mice had similarly increased PDGF-β mRNA levels when cultured under hypoxic (3% O₂) as opposed to normoxic conditions.

Next whether monocyte/macrophage-derived PDGF-β contributes to hypoxia-induced PH was assessed. Previously, it was found that tamoxifen treatment of Csflr-Mer-iCre-Mer, PDGF-β^{(flox/flox)} mice modestly attenuates pathological distal pulmonary arteriole muscularization (Sheik 2018), but effects on PH, RVH and myofibroblast accumulation were not studied. The inducible Csflr-Cre is highly inefficient at inducing recombination (Qian Nature. 2011; 475(7355):222-5; Epelman Immunity. 2014; 40(1):91-104), and herein, to bypass this inefficiency, the constitutive LysM-Cre was used to delete PDGF-β (Fig. S3A). On the PDGF-β^{(flox/flox)} background, mice also carrying LysM-Cre have attenuated distal muscularization and PH with 21-day hypoxia exposure in comparison to those with no Cre (FIG. 2C, D). When comparing the Fulton index of LysM-Cre, PDGF-β^{(flox/flox)} to that of PDGF-β^{(flox/flox)} mice, there was a trend toward reduction with hypoxia and increase with normoxia, but these differences did not reach statistical significance (FIG. 2E). However, when the Fulton index differences between hypoxia and normoxia values were stratified by genotype, there was a significant 46±7% reduction in this difference for LysM-Cre, PDGF-β^{(flox/flox)} mice (FIG. 2F). Finally, with myeloid cell PDGF-β deletion, myofibroblasts were reduced by ˜60% at both 3 and 21 days of hypoxia (FIGS. 2G, H, S4A, B). Thus, myeloid cell-derived PDGF-B is an important player in hypoxia-induced pulmonary vascular remodeling and PH.

### LysM-Cre-Mediated Deletion of Von-Hippel Lindau Induces PDGF-β Expression and Pulmonary Vascular Remodeling in Normoxia

Given the critical role of myeloid cell-derived PDGF-B in the pathogenesis of PH, the mechanisms underlying hypoxia-induced PDGF-β expression by this cell type were evaluated. Hypoxia-inducible factors (HIFs) are heterodimers of HIF1-β and a HIFα isoform, either HIF1-α or HIF2-α. In mice exposed to hypoxia, EC HIF regulates cell autonomous PDGF-β expression as well as distal muscularization and PH. Using oxygen as a substrate, HIFα undergoes proline hydroxylation, a modification that facilitates binding to von-Hippel Lindau (VHL)-E3 ubiquitin ligase and ultimately proteosomal-mediated degradation. Thus, HIFα accumulates when oxygen is scare or when the relevant ubiquitination-degradation pathway is inhibited, such as by Vhl deletion. Under normoxic conditions, in comparison to Vhl^{(flox/flox)} mice, LysM-Cre, Vhl^{(flox/flox)} mice have reduced Vhl and increased Hif1a, Hif2a and PDGF-levels in BALF cells (FIGS. 3A, S3D-F). Furthermore, Vhl deletion in myeloid cells induces distal muscularization, PH and RVH in normoxia (FIG. 3B-C) as well as lung macrophage accumulation (FIG. 3D).

Whether Vhl deletion potentiates the effects of a relatively brief (7 day) exposure to hypoxia was then evaluated. At this time point, Vhl^{(flox/flox)} mice carrying LysM-Cre have BALF cell PDGF-β mRNA levels that are robustly increased at 7.6±1.2-fold relative to that of mice lacking Cre. Furthermore, Vhl deletion in LysM⁺ cells induces markedly enhanced distal muscularization as well as increased RVSP and RVH following brief hypoxia exposure.

### Myeloid Cell HIFα Regulates PDGF-β Expression and Hypoxia-Induced Distal Muscularization, RVH and PH

To complement the experiments that delete Vhl and thus, induce the HIF pathway, studies that delete Hif1a or Hif2a in LysM⁺ cells were pursued. First, a time course of hypoxia exposure of wild type mice revealed HIF1-α and HIF2-α upregulation in BALF cells by hypoxia day 3 (FIGS. 4A, 5A). At this time point, mice on the Hif1a^{(flox/flox)} or Hif2a^{(flox/flox)} background and also carrying LysM-Cre have reduced levels of PDGF-β and either Hif1a or Hif2a, respectively, in BALF cells in comparison to mice lacking Cre (FIGS. 4B, 5B). In addition, accumulation in the lung of cells expressing the macrophage marker CD64 and of myofibroblasts is substantially reduced with Hif1a or Hif2a deletion (FIGS. 4C-D, 5C-D). Moreover, analysis at hypoxia day 21 revealed that LysM-Cre mice carrying Hif1a^{(flox/flox)} or Hif2a (flox flox) have attenuated distal pulmonary arteriole muscularization, RVSP and Fulton index (FIGS. 4E-F, 5E-F). Thus, taking PDGF-β, Vhl, Hif1a and Hif2a deletion experiments together, the results suggest that PDGF-B expression by myeloid cells is modulated cell autonomously by both HIFα isoforms and is a key factor regulating pulmonary vascular remodeling and PH.

### Macrophage-Derived PDGF-B is Increased in PAH Patients and Induces SMC Proliferation and Migration

Given the prominent role of macrophages and myeloid-derived PDGF-B in pathological lung muscularization in mice, we next sought to extrapolate these findings to human PAH patients. Initially, PDGF-β levels from human macrophages were analyzed. The peripheral blood mononuclear cell fraction was isolated from fresh whole blood of control humans by Ficoll column centrifugation and enriched for monocytes by adherence to plastic. Adherent cells were incubated with macrophage colony-stimulating factor to differentiate them to macrophages, and exposure of macrophages to hypoxia (3% O₂) as opposed to normoxia for 12 h induced a 2.6±0.6-fold increase in PDGF-β transcript levels (FIG. 6A). As strong evidence of the clinical relevance of this work, PDGF-β levels of macrophages differentiated from circulating monocytes of IPAH and SSc-PAH patients were enhanced by 5.1±1.8 and 10.7±4.8-fold, respectively, in comparison that of control humans (FIG. 6B).

The effect of medium conditioned by macrophages from PAH patients on hPASMC proliferation and the role of PDGF-B in this medium were evaluated. hPASMCs were cultured for 24 h in medium conditioned by newly differentiated macrophages, and BrdU was added for the final 10 h of this incubation. The percent of cells (propidium iodide [PI]⁺ nuclei) that were proliferative (i.e., BrdU) relative to control was determined (FIG. 6C). For medium conditioned by macrophages derived from IPAH and SSc-PAH patients, there was a relative increase in hPASMC proliferation by 4.6±0.3 and 7.0±1.9-fold, respectively. To evaluate the contribution of PDGF-B to these effects, macrophage conditioned medium was incubated with anti-PDGF-B blocking antibody or IgG control for 1 h prior to adding to hPASMCs. For macrophages derived from control patients, hPASMC proliferation was not changed by anti-PDGF-B pre-treatment whereas this pre-treatment significantly inhibited hPASMC proliferation-induced by medium conditioned by IPAH or SSc-PAH macrophages (FIG. 6D).

Next, a similar approach was used to investigate the effect of macrophage conditioned medium and PDGF-B therein on hPASMC migration. hPASMC migration from the top of a Boyden chamber towards the bottom chamber containing conditioned medium pre-treated, as in the proliferation studies, was assessed with an anti-PDGF-B or IgG control antibody. For IgG control pre-treatment, conditioned medium from IPAH or SSc-PAH macrophages induced migration relative to that from control macrophages by 3.0±0.8 or 4.2±0.8-fold, respectively. Furthermore, in comparison to IgG pre-treatment, anti-PDGF-B pre-treatment reduced hPASMC migration with IPAH or SSc-PAH macrophage conditioned medium by ˜40-50%. In contrast, PDGF-B pre-treatment of conditioned medium from control humans did not affect hPASMC migration.

Nanoparticle Delivery of siPDGF-β Attenuates Hypoxia-Induced PH

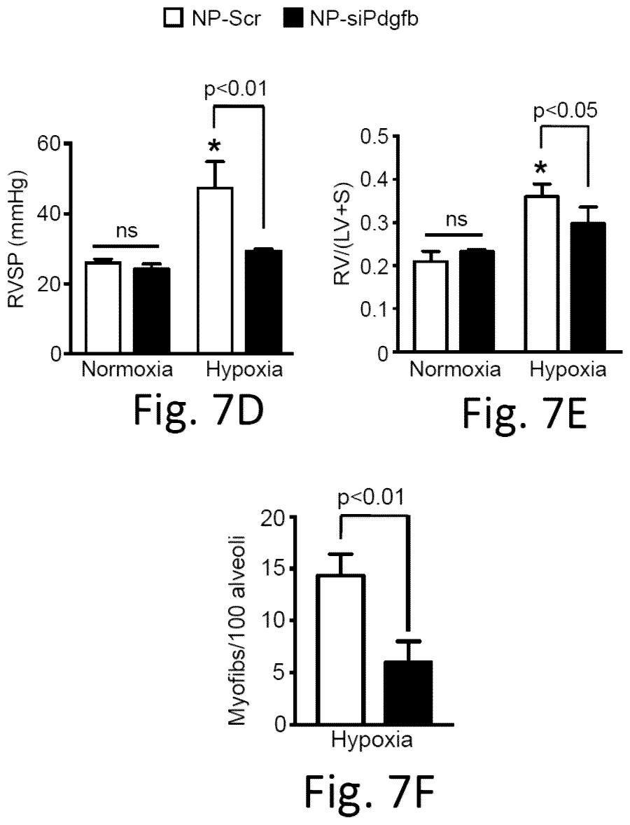

After demonstrating the importance of myeloid-derived PDGF-B in experimental PH and the inductive effects of PDGF-B from macrophages of PAH patients on hPASMCs, this ligand in lung macrophages was pharmacologically downregulated by delivering nanoparticles formed from a poly(amine-co-ester) [PACE] polymer and PDGF-β siRNA. In prior studies, it was shown that similar nanoparticles are capable for sustained silencing of protein expression in cells that internalize the particles. First, 400 or 200 nm diameter nanoparticles composed of acid-ended (poly(pentadecalactone-co-n-methyldiethanolamineco-sebacate) with 50% lactone (PPMS-50COOH) loaded with the dye DiD were orotracheally administered to wild type mice, and 12 hours later, flow cytometric analysis was used to evaluate the uptake by lung cells expressing the macrophage marker CD64⁺ (FIG. 7A). For both 400 and 200 nm diameter nanoparticles, the vast majority of CD64 cells were DiD-labeled (>99% in BALF and ˜92% in residual lung. Similarly, the percent of DiD-labeled cells that were CD64 was high and equivalent for 400 and 200 nm diameter particles (95±1% and 93±3%, respectively) in BALF; however, in the residual lung, these percentages were 86±1% for 400 nm particles and dropped down to 62±1% for 200 nm particles (FIG. 7C). Thus, all further experiments were conducted with 400 nm diameter nanoparticles. To confirm uptake, isolated BALF cells were cultured with DiD-loaded nanoparticles for 6 h, and these cells displayed perinuclear fluorescence.

Whether nanoparticles loaded with siRNA targeting PDGF-β ameliorated the effects of hypoxia exposure on the murine lung was then evaluated. A PDGF-β siRNA oligonucleotide was used that when transfected into BALF cells reduced PDGF-β levels by 91±1% in comparison to Scr RNA treatment. Nanoparticles loaded with this siPDGF-β or Scr RNA were administered orotracheally at the onset of hypoxia and twice per week for up to 21 days of hypoxia exposure. At hypoxia day 3 or 21, the percent of cells in the whole lung that were CD64 LysG-macrophages did not differ between mice treated with the two nanoparticle types (FIG. 7B-C). The effect of siPDGF-β-nanoparticles on macrophage PDGF-β RNA levels at day 3, the time of maximal PDGF-β levels was then determined (see FIG. 2A, B). Nanoparticles loaded with siPDGF-β reduced lung macrophage PDGF-β levels by 86±11% (FIG. 7C). Finally, siPDGF-β-nanoparticle treatment during the 21-day hypoxia exposure markedly attenuated distal pulmonary arteriole muscularization, PH, RVH and accumulation of myofibroblasts (FIG. 7D-F).

### Discussion

Expansion of the SMC lineage is increasingly recognized as a key factor in diverse cardiovascular diseases; however, in these pathological contexts as well as during normal vascular development, the understanding of the non-cell autonomous regulation of SMCs by cell types beyond ECs is rudimentary. Phagocytes, including macrophages, play fundamental roles in both the innate immune system and the pathogenesis of diverse cardiovascular diseases, including PH. During the embryonic period, fetal macrophage precursors are recruited to the normal lung and differentiate into macrophages, and subsequently, these resident macrophages are maintained by local proliferation. In contrast, during PH, increased monocytes are found in the pulmonary vasculature and perivascular regions and give rise to lung macrophages. Although vascular SMCs and lung macrophages are undoubtedly important cell types in PH, a critical unresolved issue is whether and how lung macrophages regulate SMCs in this context. Herein, our studies with mouse models of PH and human macrophages from IPAH and SSc-PAH patients demonstrate that macrophage-derived PDGF-B induces pathological SMC expansion and PH and thereby, establish macrophage-derived PDGF-B as a key factor in this paradigm. Moreover, our findings with nanoparticle-derived PDGF-β siRNA put forth an intriguing therapeutic approach.

Intratracheally administered clodronate-containing liposomes has previously been shown to deplete alveolar macrophages and reduce hypoxia-induced PH and RVH in rats. Herein, we demonstrate that such treatment in mice reduces macrophages in the residual lung as well as BALF and also attenuates distal muscularization and hemodynamic changes (FIG. 1). Although this approach is beneficial in the short-term, chronically depleting macrophages is not feasible given their integral role in innate immunity. Thus, a preferred strategy is to target specific macrophage-derived gene products.

Along these lines, PDGF is widely implicated in the pathogenesis of PH. In human IPAH, mRNA levels of ligands PDGFA, PDGF-B and receptors PDGFRA and PDGFRB are upregulated in small pulmonary vessels, and PDGFR-β protein is increased in whole lung lysates. Mice with a knock-in mutant Pdgfrb encoding a protein that is defective in mediating downstream PI3K and PLC-gamma signaling have blunted hypoxia-induced pulmonary vascular remodeling, PH and RVH. In a fetal lamb model in which PH is induced by intrauterine partial ligation of the ductus arteriosus, infusion of an anti-PDGF-B aptamer into the pulmonary artery reduces the severity of pulmonary vascular remodeling by one-half and RVH by two-thirds. Moreover, global PDGF-β^{(flox/flox)} mice lack hypoxia-induced distal pulmonary arteriole SMCs whereas EC-specific deletion of PDGF-β reduces but does not entirely prevent distal muscularization. Herein, we demonstrate that upon exposing mice to hypoxia, expression of PDGF-β by alveolar and residual lung macrophages is markedly upregulated (by hypoxia day 3) and LysM-Cre, PDGF-β^{(flox/flox)} mice have substantially attenuated distal muscularization and PH. Interestingly, in these hypoxic mice, there is a trend to a reduction in RVH, but it does not reach statistical significance likely because of a trend towards increased RV weight ratio under normoxia in these mutants. Indeed, the hypoxia-induced increase in RVH stratified by genotype is reduced by ˜50% with PDGF-β deletion. The explanation for the trend towards enhanced RV weight ratio under basal conditions is not clear, but we suggest that myeloid cell derived PDGF-B may limit RV mass during normal development and/or maintenance.

This data indicates that lung macrophage-derived PDGF-B plays an important role in PH; however, the regulation of PDGF-B expression in this cell type is poorly understood. With exposing mice to hypoxia, lung ECs increase PDGF-β levels in a HIF1a dependent manner, and herein, it was found that myeloid cell Hif1a or Hif2a deletion reduces PDGF-β levels in lung macrophages compared to control mice. The data indicate that Hif1a deletion in myeloid cells is protective against hypoxia-induced PH. In addition, LysM-Cre, Hif2a^{(flox/flox)} mice are protected from Schistosoma-induced PH, and the results indicate that these mice similarly have attenuated hypoxia-induced PH. The complementary HIF gain-of-function studies (i.e., myeloid Vhl deletion) suggest that lung macrophage HIF is sufficient to induce cell autonomous PDGF-β expression, distal muscularization, PH and RVH under normoxic conditions (FIG. 3). Thus, it is believed that HIF-induced PDGF-B in macrophages is integral to the hypoxic response of vascular remodeling and hemodynamic changes.

These findings demonstrate that similar to distal arteriole muscularization, lung macrophages induce accumulation of alveolar myofibroblasts in the hypoxic lung (FIG. 1), and myeloid-derived PDGF-β, Hif1a and Hif2a are critical for this process (FIGS. 2, 4, 5). Lung myofibroblasts play a key role in alveolar septal formation during normal alveologenesis in early postnatal mice, and subsequently, in the adult lung, these cells are very rare. In fibrotic disease, myofibroblasts are implicated in generating much of the excess extracellular matrix, and macrophages secrete profibrotic factors that recruit and activate myofibroblasts. In contrast, the role of monocytes/macrophages in regulating hypoxia-induced alveolar myofibroblasts has not been previously reported. PDGFR-β⁺ cells give rise to over 40% of hypoxia-induced myofibroblasts in the lung (R. Chandran, I. Kabir, A. Sheikh, ELH and DMG, unpublished data) whereas SMA cells are the source of only ˜20%. These results are in line with other studies suggesting that lung pericytes, which are PDGFR-β⁺SMA⁻, are an important cell type in PH.

Approximately 10⁻¹⁵% of patients with SSc develop PAH, and PAH is the leading cause of mortality in these patients. Indeed, the three year survival is estimated at only 49% for SSc-PAH in comparison to 84% for IPAH patients. One factor contributing to this heightened lethality is the muted response to standard anti-PAH treatments in SSc-PAH compared to IPAH patients. In addition, anti-PDGFR-β immunohistochemical staining is enhanced in the small vessels of patients with SSc-PAH in comparison to those with IPAH. The number of circulating monocytes does not differ between these PAH patient populations; however, the results indicate that in macrophages derived from these monocytes, in comparison to control humans, PDGF-B levels are more enhanced in SSc-PAH than in IPAH patients. Additionally, macrophages from these two classes of PAH patients induce SMC proliferation and migration in a largely PDGF-B-dependent manner. A study published 25 years ago reported that PDGF-B protein level is increased in the BALF of general SSc patients (i.e., patients not evaluated for PH) compared to that of controls. Thus, a strategy targeting macrophage-derived PDGF-B may have efficacy in PAH.

Imatinib is a tyrosine kinase inhibitor with activity against BCR-ABL, c-KIT, PDGFR-α and -β with applications in cancers. Daily injections of imatinib reverses pulmonary vascular remodeling, PH and RVH due to monocrotaline in rats or chronic hypoxia in mice. Unfortunately, these positive results did not extrapolate to PAH patients in the Imatinib in Pulmonary Arterial Hypertension, a Randomized Efficacy Study (IMPRES). Overall, 94% of patients discontinued this oral imatinib study and serious and unexpected adverse effects were common, including subdural hematoma. Notably, however, patients in IMPRES that were able to remain on imatinib for a long duration showed improved functional class and 6 minute walk distance. These results further emphasize the need for anti-PH therapy that targets a specific pathway (e.g., PDGF-B-mediated) in a specific cell type (e.g., macrophages) in the lung.

Orotracheally administered PPMS polymer-formulated nanoparticles loaded with siRNA targeting PDGF-β substantially downregulate macrophage-derived PDGF-β, preventing hypoxia-induced distal pulmonary arteriole muscularization, PH and RVH. These nanoparticles are specifically and broadly phagocytosed by lung macrophages. Previous studies have shown that intratracheal or intravenous delivery of nanoparticles carrying agents with efficacy in human PAH, including prostacyclin analogues and sildanefil, attenuates PH in experimental rodent models. The only prior report of nanoparticle-mediated RNA interference in this context demonstrated that intravenous delivery of antisense oligonucleotide microRNA (antimiR)-145, which aims to directly target SMCs, mitigates hypoxia/Sugen-5416-induced PH in rats; however, in addition to the lung, this antimiR accumulates in the liver, spleen and kidney. The approach herein of orotracheally administering nanoparticle loaded siRNA is advantageous as it specifically and potently targets a select gene product in lung macrophages and thereby, promises to limit untoward effects. Furthermore, PPMS polymer-formulated nanoparticles are non-toxic and biodegradable and protect their cargo from degradation.

Taken together, the studies with an experimental model as well as cells isolated form human PAH patients demonstrate that HIF-regulated expression of PDGF-B by macrophages plays a major role in SMC remodeling, PH and RVH. Furthermore, nanoparticle-mediated silencing of PDGF-β in lung macrophages is a therapeutic strategy that warrants intense further investigation.

### SUMMARY

The results show that PACE nanoparticles provided selective uptake in pulmonary macrophages and monocytes following oral (or pulmonary) administration.

The results also establish that this method of delivery of an inhibitor of PDGE-β was effective in treating PH.

FIGS. 1A-1F show that macrophages from BALF and residual lung increase with hypoxia exposure. Mice were exposed to normoxia or hypoxia (FiO₂ 10%) for 0-21 days. CD64+Ly6G− macrophages were isolated by FACS from BALF and subjected to qRT-PCR for PDGF-β. Similarly, CD64+Ly6G− macrophages were isolated from the residual lung and PDGF-β mRNA levels were evaluated.

FIGS. 2A-2F show that macrophage depletion is protective against pulmonary hypertension. Mice were exposed to normoxia or hypoxia (FiO₂ 10%) for 21 days and concomitantly received orotracheal liposomes loaded with clodronate or vehicle two times per week. Clodronate treatment reduced distal arterial muscularization, as shown by right ventricle systolic pressure (RVSP; equivalent to pulmonary artery systolic pressure) and the Fulton index in hypoxic mice.

As shown by FIGS. 3A-3D, 4A-4F, and 5A-5F, hypoxia induces PDGF-β in macrophages of BALF and residual lung. In LysM-Cre lineage, PDGF-β or Hif2a deletion attenuates hypoxia-induced distal muscularization and PH, and Vhl deletion induces spontaneous PH.

PDGF-β and Hif2a deletion in myeloid cells protects against PH while Vhl deletion leads to PH under normoxic conditions. Mice were exposed to hypoxia or normoxia as indicated. Lung sections with distal arterioles from mice carrying no Cre or LysM-Cre and floxed alleles for PDGF-β, Hif2a or Vhl were stained for markers of SMCs (alpha-smooth muscle actin [SMA]) and endothelial cells (ECs; MECA-32).

Myeloid cells from human PH patients have increased PDGF-β levels which induces SMC proliferation and migration.

FIGS. 6A-6E show that macrophage-derived PDGF-B in idiopathic and scleroderma PAH patients promotes SMC proliferation and migration. Human macrophages were cultured under normoxia or hypoxia (3% O2) for 12 h, and then PDGF-β mRNA in macrophages from control and PAH patients were measured by qRT-PCR. The BrdU assay was performed on human pulmonary artery SMCs cultured with patient or control culture media (CM). Anti-PDGF-B blocking Ab or control IgG was added to CM. A migration assay with SMCs added to top of Boyden chamber and CM on the bottom with either anti-PDGF-B Ab or IgG was used to quantify migrated cells relative to controls.

FIGS. 7A-7F show that PACE Nanoparticle (NP)-mediated PDGF-β knockdown in myeloid cells attenuates PH. Mice were exposed to normoxia or hypoxia and concomitantly received NP loaded with scrambled (Scr) RNA or PDGF-βtargeted siRNA. CD64+Ly6G− macrophages isolated by FACS were subjected to qRT-PCR for PDGF-β mRNA. Lung sections with distal arterioles were stained for SMA and CD31 (EC marker). RVSP and Fulton index were measured. Nanoparticle-delivered siPDGF-β to lung macrophages attenuates hypoxia-induced distal muscularization, PH and RVH.

Accordingly, the results establish that:
1. PH can be treated or prevented by administration of a PDGF-β to the lung; and
2. One can achieve selective uptake of agent in pulmonary macrophages and monocytes using nanoparticles formed of PACE polymers.

Unless defined otherwise, all technical and scientific terms used herein have the same meanings as commonly understood by one of skill in the art to which the invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A delivery formulation for selective delivery to pulmonary immune cells comprising macrophages and monocytes, the delivery formulation comprising:
(i) nanoparticles having an averaged diameter between 100 and 500 nm and comprising a polymer having a plurality of monomeric units: 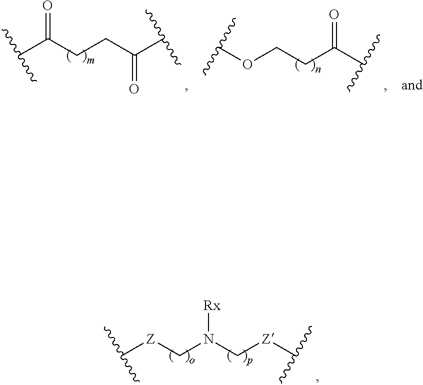
wherein n is independently at each occurrence an integer from 1-30, m, o, and p are independently at each occurrence integers from 1-20, Rx is independently at each occurrence hydrogen, substituted or unsubstituted alkyl, or substituted or unsubstituted aryl, or substituted or unsubstituted alkoxy, Z and Z′ are independently at each occurrence O or NR′, wherein R′ is hydrogen, substituted or unsubstituted alkyl, or substituted or unsubstituted aryl,
wherein the polymer comprises an end group at one or both ends selected from substituted alkyl comprising a primary amine group, a secondary amine group, a tertiary amine group, or combinations thereof; and
(ii) an agent for the treatment or diagnosis of pulmonary hypertension, wherein the agent is encapsulated within the nanoparticles, dispersed within a polymer matrix that forms the nanoparticles, covalently or non-covalently associated with a surface of the nanoparticles, or combinations thereof,
wherein selective delivery to the pulmonary immune cells occurs when the formulation is administered via oral or pulmonary administration.

2. The formulation of claim 1, wherein the end group further comprises a hydroxyl group, and/or a linker moiety selected from —NH—, —O—, —C(O)NH—, and —C(O)O—.

3. The formulation of claim 1, wherein the nanoparticles have a diameter between 200 nm and 400 nm.

4. The formulation of claim 1, wherein the nanoparticles have a uniform size distribution within less than about 20% standard deviation of 400 nm.

5. The formulation of claim 1, wherein the polymer has a weight average molecular weight between about 2,000 Daltons and 20,000 Daltons, as measured by gel permeation chromatography using narrow polydispersity polystyrene standards.

6. The formulation of claim 1, wherein the agent is an inhibitor of PDGF-β.

7. The formulation of claim 1, wherein the agent is for diagnosis of pulmonary hypertension.

8. The formulation of claim 6, wherein the inhibitor is an siRNA inhibitor of PDGF-β.

9. The formulation of claim 7, wherein the agent is a protein or peptide, sugar or carbohydrate, lipid, lipoprotein, or lipopolysaccharide, nucleic acid molecule, or small molecule having a molecular weight of less than 2000 Daltons.

10. The formulation of claim 1, wherein the formulation is formulated for administration as an aerosol, for instillation, in a nebulizer, in an inhaler, in a ventilator or breathing mask, or as a dry powder.

11. A method for treating a disease or condition in a subject in need thereof comprising administering via oral or pulmonary administration an effective amount of a formulation comprising:
(i) nanoparticles having an average diameter between 100 and 500 nm and comprising a polymer comprising a plurality of monomeric units: 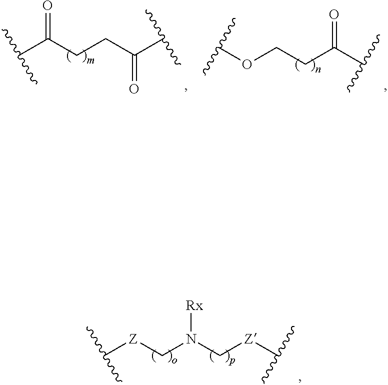
and one or more end groups each independently at each occurrence comprising a hydroxyl group, a primary amine group, a secondary amine group, a tertiary amine group, or combinations thereof,
wherein n is independently at each occurrence an integer from 1-30, m, o, and p are independently at each occurrence integers from 1-20, Rx is independently at each occurrence hydrogen, substituted or unsubstituted alkyl, or substituted or unsubstituted aryl, or substituted or unsubstituted alkoxy, Z and Z′ are independently at each occurrence O or NR′, wherein R′ is hydrogen, substituted or unsubstituted alkyl, or substituted or unsubstituted aryl; and
(ii) an agent for the treatment or diagnosis of pulmonary hypertension, wherein the agent is encapsulated within the nanoparticles, dispersed within a polymer matrix that forms the nanoparticles, covalently or non-covalently associated with a surface of the nanoparticles, or combinations thereof,
wherein the agent is selectively delivered to pulmonary immune cells comprising macrophages and monocytes.

12. The method of claim 11, wherein the end group further comprises a linker moiety selected from —NH—, —O—, —C(O)NH—, and —C(O)O—.

13. The method of claim 11, wherein the nanoparticles have a diameter between 200 nm and 400 nm, or have a uniform size distribution within less than about 20% standard deviation of 400 nm.

14. The method of claim 11, wherein the polymer has a weight average molecular weight between about 2,000 Daltons and 20,000 Daltons, as measured by gel permeation chromatography using narrow polydispersity polystyrene standards.

15. The method of claim 11, wherein the agent is for diagnosis of pulmonary hypertension, or a protein or peptide, sugar or carbohydrate, lipid, lipoprotein, lipopolysaccharide, nucleic acid molecule, or small molecule having a molecular weight of less than 2000 Daltons.

16. The method of claim 11, wherein the agent is an siRNA inhibitor of PDGF-β.

17. The method of claim 11, wherein the formulation is formulated for administration as an aerosol, for instillation, in a nebulizer, in an inhaler, in a ventilator or breathing mask, or as a dry powder, and wherein the agent is in an amount for local delivery of the agent to the immune cells of the pulmonary system, not systemically.

18. The method of claim 11, wherein the formulation is administered every day, every 2 days, or every 3 days, for a week, two weeks, 3 weeks, or 4 weeks.

19. The method of claim 11, wherein the disease or condition is pulmonary hypertension, congestive heart failure, lung fibrosis, lung cancer, or a viral disease.

20. The method of claim 11, wherein the subject has or is at risk of developing acute respiratory distress syndrome.
